(19) United States
(12) Patent Application Publication
(10) Pub. No.: US 2005/0143578 A1
(43) Pub. Date: Jun. 30, 2005

(54) Compounds and methods

(75) Inventors: Lara S. Kallander, King of Prussia, PA (US); M. Dominic Ryan, Littleton, MA (US); Scott K. Thompson, King of Prussia, PA (US)

Correspondence Address: GLAXOSMITHKLINE;Corporate Intellectual Property - UW2220, P.O. Box 1539, King of Prussia, PA, 19406-0939, US

(73) Assignee: SmithKline Beecham Corporation

(21) Appl. No.: 11/063,166

(22) Filed: Feb. 22, 2005

Related U.S. Application Data
(63) Continuation of application No. 10/492,218, filed on Apr. 9, 2004, now abandoned.
(60) Provisional application No. 60/328,753, filed on Oct. 12, 2001.

(51) Int. Cl. C07D 43/04

(52) U.S. Cl. 544/329; 546/268.1

(57) ABSTRACT

Disclosed are compounds of the formula: 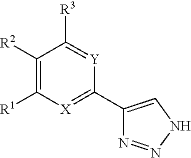 wherein the formula variables are as defined herein. The compounds of this invention are non-peptide, reversible inhibitors of type 2 methionine aminopeptidase. Also disclosed is the use of such compounds in treating conditions mediated by angiogenesis, such as cancer, haemangioma, proliferative retinopathy, rheumatoid arthritis, atherosclerotic neovascularization, psoriasis, ocular neovascularization and obesity.

### FIELD OF THE INVENTION

Compounds of this invention are non-peptide, reversible inhibitors of type 2 methionine aminopeptidase, useful in treating conditions mediated by angiogenesis, such as cancer, haemangioma, proliferative retinopathy, rheumatoid arthritis, atherosclerotic neovascularization, psoriasis, ocular neovascularization and obesity.

### BACKGROUND OF THE INVENTION

In 1974, Folkman proposed that for tumors to grow beyond a critical size and to spread to form metastases, they must recruit endothelial cells from the surrounding stroma to form their own endogenous microcirculation in a process termed angiogenesis-(Folkman J. (1974) Adv Cancer Res. 19; 331). The new blood vessels induced by tumor cells as their life-line of oxygen and nutrients also provide exits for cancer cells to spread to other parts of the body. Inhibition of this process has been shown to effectively stop the proliferation and metastasis of solid tumors. A drug that specifically inhibits this process is known as an angiogenesis inhibitor.

Having emerged as a promising new strategy for the treatment of cancer, the anti-angiogenesis therapy (“indirect attack”) has several advantages over the “direct attack” strategies. All the “direct attack” approaches such as using DNA damaging drugs, antimetabolites, attacking the RAS pathway, restoring p53, activating death programs, using aggressive T-cells, injecting monoclonal antibodies and inhibiting telomerase, etc., inevitably result in the selection of resistant tumor cells. Targeting the endothelial compartment of tumors as in the “indirect attack”, however, should avoid the resistance problem because endothelial cells do not exhibit the same degree of genomic instability as tumor cells. Moreover, anti-angiogenic therapy generally has low toxicity due to the fact that normal endothelial cells are relatively quiescent in the body and exhibit an extremely long turnover. Finally since the “indirect attack” and “direct attack” target different cell types, there is a great potential for a more effective combination therapy.

More than 300 angiogenesis inhibitors have been discovered, of which about 31 agents are currently being tested in human trials in treatment of cancers (Thompson, et al., (1999) J Pathol 187, 503). TNP-470, a semisynthetic derivative of fumagillin of Aspergillus fuigatus, is among the most potent inhibitors of angiogenesis. It acts by directly inhibiting endothelial cell growth and migration in vitro and in vivo (Ingber et al. (1990) Nature 348, 555). Fumagillin and TNP-470, have been shown to inhibit type 2 methionine aminopeptidase (hereinafter MetAP2) by irreversibly modifying its active site. The biochemical activity of fumagillin analogs has been shown to correlate to their inhibitory effect on the proliferation of human umbillical vein endothelial cells (HUVEC). Although the mechanism of the selective action of fumagillin and related compounds on MetAP2-mediated endothelial cell cytostatic effect has not yet been established, possible roles of MetAP2 in cell proliferation have been suggested.

First, hMetAP-2-catalyzed cleavage of the initiator methionine of proteins could be essential for releasing many proteins that, after myristoylation, function as important signaling cellular factors involved in cell proliferation. Proteins known to be myristoylated include the src family tyrosine kinases, the small GTPase ARF, the HIV protein nef and the α subunit of heterotrimeric G proteins. A recently published study has shown that the myristoylation of nitric oxide synthase, a membrane protein involved in cell apoptosis, was blocked by fumagillin (Yoshida, et al. (1998) Cancer Res. 58(16), 3751). This is proposed to be an indirect outcome of inhibition of MetAP2-catalyzed release of the glycine-terminal myristoylation substrate. Alternatively, MetAP enzymes are known to be important to the stability of proteins in vivo according to the “N-end rule” which suggests increased stability of methionine-cleaved proteins relative to their N-terminal methionine precursors (Varshavsky, A (1996) Proc. Natl. Acad. Sci. U.S.A. 93, 12142). Inhibition of hMetAP2 could result in abnormal presence or absence of some cellular proteins critical to the cell cycle.

Methionine aminopeptidases (MetAP) are ubiquitously distributed in all living organisms. They catalyze the removal of the initiator methionine from newly translated polypeptides using divalent metal ions as cofactors. Two distantly related MetAP enzymes, type 1 and type 2, are found in eukaryotes, which at least in yeast, are both required for normal growth; whereas only one single MetAP is found in eubacteria (type 1) and archaebacteria (type 2). The N-terminal extension region distinguishes the methionine aminopeptidases in eukaryotes from those in procaryotes. A 64-amino acid sequence insertion (from residues 381 to 444 in hMetAP2) in the catalytic C-terminal domain distinguishes the MetAP-2 family from the MetAP-1 family. Despite the difference in the gene structure, all MetAP enzymes appear to share a highly conserved catalytic scaffold termed “pita-bread” fold (Bazan, et al. (1994) Proc. Natl. Acad. Sci. U.S.A. 91, 2473), which contains six strictly conserved residues implicated in the coordination of the metal cofactors.

Mammalian type 2 methionine aminopeptidase has been identified as a bifunctional protein implicated by its ability to catalyze the cleavage of N-termiinal methionine from nascent polypeptides (Bradshaw, et al (1998) Trends Biochem. Sci. 23, 263) and to associate with eukaryotic initiation factor 2α (eIF-2α) to prevent its phosphorylation (Ray, et al. (1992) Proc. Natl. Acad. Sci. U.S.A. 89, 539). Both the genes of human and rat MetAP2 were cloned and have shown 92% sequence identity (Wu,. et al. (1993) J. Biol. Chem. 268, 10796; Li, X. & Chang, Y.-H. (1996) Biochem. & Biophys. Res. Comm. 227, 152). The N-terminal extension in these enzymes is highly charged and consists of two basic polylysine blocks and one aspartic acid block, which has been speculated to be involved in the binding of eIF-2α (Gupta, et al. (1993) in Translational Regulation of Gene Expression 2 (ilan, J., Ed.), pp. 405-431, Plenum Press, New York).

The anti-angiogenic compounds, fumagillin and its analogs, have been shown to specifically block the exo-aminopeptidase activity of hMetAP2 without interfering with the formation of the hMetAP2: eIF2α complex (Griffith, et al., (1997) Chem. Biol. 4, 461; Sin, et al. (1997) Proc. Natl. Acad. Sci. U.S.A. 94, 6099). Fumagillin and its analogs inactivate the enzymatic activity of hMetAP2 with a high specificity, which is underscored by the lack of effect of these compounds on the closely related type 1 methionine aminopeptidase (MetAP1) both in vitro and in vivo in yeast (Griffith, et al., (1997) Chem. Biol. 4, 461; Sin, et al. (1997) Proc. Natl. Acad. Sci. U.S.A. 94, 6099). The extremely high potency (IC₅₀<1 nM) of these inhibitors appears to be due to the irreversible modification of the active site residue, His231, of hMetAP2 (Liu, et al. (1998) Science 282, 1324). Disturbance of MetAP2 activity in vivo impairs the normal growth of yeast (Griffith, et al., (1997) Chem. Biol. 4, 461; Sin, et al. (1997) Proc. Natl. Acad. Sci. U.S.A. 94, 6099; In-house data) as well as Drosophila (Cutforth & Gaul (1999) Mech. Dev. 82, 23). Most significantly, there appears to be a clear correlation between the inhibition effect of fumagillin related compounds against the enzymatic activity of hMetAP2 in vitro and the suppression effect of these compounds against tumor-induced angiogenesis in vivo (Griffith, et al., (1997) Chem. Biol. 4, 461).

Cancer is the second leading cause of death in the U.S., exceeded only by heart disease. Despite recent successes in therapy against some forms of neoplastic disease, other forms continue to be refractory to treatment. Thus, cancer remains a leading cause of death and morbidity in the United States and elsewhere (Bailar and Gornik (1997) N. Engl. J. Med. 336, 1569). Inhibition of hMetAP2 provides a promising mechanism for the development of novel anti-angiogenic agents in the treatment of cancers. It has now been discovered that compounds of Formulae (I) and (II) are effective inhibitors of hMetAP2, and thus would be useful in treating conditions mediated by hMetAP2.

### SUMMARY OF THE INVENTION

In one aspect, the present invention is directed to a compound of Formula (I): 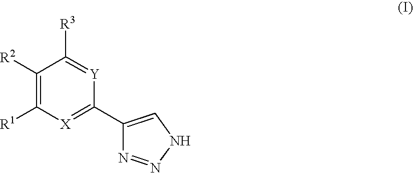
wherein:
X and Y are each independently selected from the group consisting of N, CH and CR, provided that X and Y are not both CR;
R is selected from the group consisting of: halogen, C₁-C₆ alkyl, C₁-C₆ haloalkyl, C₁-C₆ alkoxy, C₁-C₆ haloalkoxy, Ph-C₀-C₆ alkoxy, Het-C₀-C₆ alkoxy, hydroxyl, amino, (Ph-C₁-C₄ alkyl)(C₁-C₄ alkyl)N—, (Ph-C₁-C₄ alkyl)HN—, (Het-C₁-C₄ alkyl)(C₁-C₄ alkyl)N—, (Het-C₁-C₄ alkyl)HN—, (C₁-C₄ alkyl)(C₁-C₄ alkyl)N—, (C₁-C₄ alkyl)HN—, (C₁-C₄ alkyl)(C₁-C₄ alkyl)N—C₁-C₄ alkyl-, and (C₁-C₄ alkyl)HN—C₁-C₄ alkyl-;
R¹ is selected from the group consisting of: hydrogen, halogen, C₁-C₆ alkyl, C₁-C₆ haloalkyl, C₁-C₆ alkoxy, C₁-C₆ haloalkoxy, Ph-C₀-C₆ alkoxy, Het-C₀-C₆ alkoxy, (Ph-C₁-C₄ alkyl)(C₁-C₄ alkyl)N—, (Ph-C₁-C₄ alkyl)HN—, (Het-C₁-C₄ alkyl)(C₁-C₄ alkyl)N—, (Het-C₁-C₄ alkyl)HN—, (C₁-C₄ alkyl)(C₁-C₄ alkyl)N—, (C₁-C₄ alkyl)HN—, (C₁-C₄ alkyl)(C₁-C₄ alkyl)N—C₁-C₄ alkyl-, and (C₁-C₄ alkyl)HN—C₁-C₄ alkyl-;
R² and R³ are each independently selected from the group consisting of: hydrogen, halogen, C₁-C₆ alkyl, C₁-C₆ haloalkyl, C₁-C₆ alkoxy, C₁-C₆ haloalkoxy, hydroxyl, amino, (C₁-C₄ alkyl)HN— and (C₁-C₄ alkyl)(C₁-C₄ alkyl)N—;
wherein the (C₁-C₄ alkyl)N— moiety of any of the above (C₁-C₄ alkyl)(C₁-C₄ alkyl)N—, (C₁-C₄ alkyl)HN—, (C₁-C₄ alkyl)(C₁-C₄ alkyl)N—C₁-C₄ alkyl-, and (C₁-C₄ alkyl)HN—C₁-C₄ alkyl- is unsubstituted or substituted on carbon (e.g., substituted on (C₁-C₄ alkyl), not on N—) by a substituent selected from C₁-C₄ alkoxy, hydroxyl, hydroxy-C₁-C₄ alkyl-, C₁-C₄ alkylC(O)O—, C₁-C₄ alkoxyC(O)—, HOC(O)C₁-C₄ alkoxy-, and (C₁-C₄ alkoxy)C(O)C₁-C₄ alkoxy-;
and wherein said Ph and Het are unsubstituted or substituted by one or more substituents independently selected from halogen, C₁-C₄ alkyl, C₁-C₄ alkoxy, C₁-C₄ haloalkyl, C₁-C₄ haloalkoxy, hydroxyl, hydroxy-C₁-C₄ alkyl-, C₁-C₄ alkylC(O)O—, C₁-C₄ alkoxyC(O)—, HOC(O)C₁-C₄ alkoxy-, (C₁-C₄ alkoxy)C(O)C₁-C₄ alkoxy-, amino, (C₁-C₄ alkyl)HN—, (C₁-C₄ alkyl)(C₁-C₄ alkyl)N—, (C₁-C₄ alkyl)HN—C₁-C₄ alkyl-, and (C₁-C₄ alkyl)(C₁-C₄ alkyl)N—C₁-C₄ alkyl-;
or any two adjacent R, R¹, R² or R³ groups, taken together with the atoms to which they are attached, form a 6-membered carbocyclic aromatic ring, wherein said 6-membered carbocyclic aromatic ring is unsubstituted or substituted with one or more substituents independently selected from halogen, C₁-C₄ alkyl, C₁-C₄ alkoxy, C₁-C₄ haloalkyl and C₁-C₄ haloalkoxy;
provided that the compound of Formula (I) is not: 2-(1H-1,2,3-triazol4-yl)-pyridine, 4-phenyl-1H-1,2,3-triazole, 4-(1H-1,2,3-triazol-4-yl)-phenol, 4-(4-fluorophenyl)-1H-1,2,3-triazole, 4-(3-bromophenyl)-1H-1,2,3-triazole, 4-(3-bromo-4-(trifluoromethoxy)phenyl)-1H-1,2,3-triazole, 4-(3-methylphenyl)-1H-1,2,3-triazole, 4-(4-methylphenyl)-1H-1,2,3-triazole, 4-(4-bromophenyl)-1H-1,2,3-triazole, 4-(3-chlorophenyl)-1H-1,2,3-triazole, 4-(4-chlorophenyl)-1H-1,2,3-triazole, 4-(4-ethylphenyl)-1H-1,2,3-triazole, 4-(3,5-dichlorophenyl)-1H-1,2,3-triazole, 4-[4-(t-butyl)phenyl]-1H-1,2,3-triazole, 4-[4-methoxyphenyl]-1H-1,2,3-triazole, 4-(2-napthyl)-1H-1,2,3-triazole, 4-(1H-1,2,3-triazol-4-yl)-aniline, 2-chloro-4-(1H-1,2,3--triazol-4-yl)-aniline or N,N-dimethyl-4-(1H-1,2,3-triazol4-yl)-benzylamine;
or a tautomer thereof, or a pharmaceutically active salt or solvate thereof.

This invention is also directed to the use of a compound according to Formula (I) in treating conditions mediated by angiogenesis, such as cancer, haemangioma, proliferative retinopathy, rheumatoid arthritis, atherosclerotic neovascularization, psoriasis, ocular neovascularization and obesity.

In another embodiment, this invention is directed to a method of treating conditions mediated by angiogenesis, such as cancer, haemangioma, proliferative retinopathy, rheumatoid arthritis, atherosclerotic neovascularization, psoriasis, ocular neovascularization and obesity by administering a compound of Formula (II), or a pharmaceutically acceptable salt or solvate thereof: 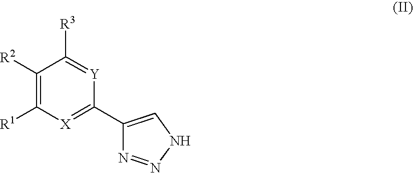
wherein:
X and Y are each independently selected from the group consisting of N, CH and CR, provided that X and Y are not both CR;
R is selected from the group consisting of: halogen, C₁-C₆ alkyl, C₁-C₆ haloalkyl, C₁-C₆ alkoxy, C₁-C₆ haloalkoxy, Ph-C₀-C₆ alloxy, Het-C₀-C₆ alkoxy, hydroxyl, amino, (Ph-C₁-C₄ alkyl)(C₁-C₄ alkyl)N—, (Ph-C₁-C₄ alkyl)HN—, (Het-C₁-C₄ alkyl)(C₁-C₄ alkyl)N—, (Het-C₁-C₄ alkyl)HN—, (C₁-C₄ alkyl)(C₁-C₄ alkyl)N—, (C₁-C₄ alkyl)HN—, (C₁-C₄ alkyl)(C₁-C₄ alkyl)N—C₁-C₄ alkyl-, and (C₁-C₄ alkyl)HN—C₁-C₄ alkyl-;
R¹ is selected from the group consisting of: hydrogen, halogen, C¹-C₆ alkyl, C¹-C₆ haloalkyl, C¹-C₆ alkoxy, C¹-C₆ haloalkoxy, Ph-C⁰-C₆ alkoxy, Het-C₀-C₆ alkoxy, (Ph-C₁-C₄ alkyl)(C₁-C₄ alkyl)N—, (Ph-C₁-C₄ alkyl)HN—, (Het-C₁-C₄ alkyl)(C₁-C₄ alkyl)N—, (Het-C₁-C₄ alkyl)HN—, (C₁-C₄ alkyl)(C₁-C₄ alkyl)N—, (C₁-C₄ alkyl)HN—, (C₁-C₄ alkyl)(C₁-C₄ alkyl)N—C₁-C₄ alkyl-, and (C₁-C₄ alkyl)HN—C₁-C₄ alkyl-;
R² and R³ are each independently selected from the group consisting of: hydrogen, halogen, C₁-C₆ alkyl, C₁-C₆ haloalkyl, C₁-C₆ alkoxy, C₁-C₆ haloalkoxy, hydroxyl, amino, (C₁-C₄ alkyl)HN— and (C₁-C₄ alkyl)(C₁-C₄ alkyl)N—;
wherein the (C₁-C₄ alkyl)N— moiety of any of the above (C₁-C₄ alkyl)(C₁-C₄ alkyl)N—, (C₁-C₄ alkyl)HN—, (C₁-C₄ alkyl)(C₁-C₄ alkyl)N—C₁-C₄ alkyl-, and (C₁-C₄ alkyl)HN—C₁-C₄ alkyl- is unsubstituted or substituted on carbon (e.g., substituted on (C₁-C₄ alkyl), not on N—) by a substituent selected from C₁-C₄ alkoxy, hydroxyl, hydroxy-C₁-C₄ alkyl-, C₁-C₄ alkylC(O)O—, C₁-C₄ alkoxyC(O)—, HOC(O)C₁-C₄ alkoxy-, and (C₁-C₄ alkoxy)C(O)C₁-C₄ alkoxy-;
and wherein said Ph and Het are unsubstituted or substituted by one or more substituents independently selected from halogen, C₁-C₄ alkyl, C₁-C₄ alkoxy, C₁-C₄ haloalkyl, C₁-C₄ haloalkoxy, hydroxyl, hydroxy-C₁-C₄ alkyl-, C₁-C₄ alkylC(O)O—, C₁-C₄ alkoxyC(O)—, HOC(O)C₁-C₄ alkoxy-, (C₁-C₄ alkoxy)C(O)C₁-C₄ alkoxy-, amino, (C₁-C₄ alkyl)HN—, (C₁-C₄ alkyl)(C₁-C₄ alkyl)N—, (C₁-C₄ alkyl)HN—C₁-C₄ alkyl-, and (C₁-C₄ alkyl)(C₁-C₄ alkyl)N—C₁-C₄ alkyl-;
or any two adjacent R, R¹, R² or R³ groups, taken together with the atoms to which they are attached, form a 6-membered carbocyclic aromatic ring, wherein said 6-membered carbocyclic aromatic ring is unsubstituted or substituted with one or more substituents independently selected from halogen, C₁-C₄ alkyl, C₁-C₄ alkoxy, C₁-C₄ haloalkyl and C₁-C₄ haloalkoxy;
or a tautomer thereof, or a pharmaceutically active salt or solvate thereof.

In another aspect, the present invention is to a method of inhibiting MetAP2 in the treatment of angiogenesis-mediated diseases, all in mammals, preferably humans, comprising administering to such mammal in need thereof, a compound of Formula (II), or a pharmaceutically active salt thereof.

In yet another aspect, the present invention is directed to a pharmaceutical composition comprising a compound of Formula (I) or Formula (II) and a pharmaceutically acceptable carrier therefor. In particular, the pharmaceutical compositions of the present invention are used for treating MetAP2-mediated diseases.

In a further aspect, the present invention is to novel intermediates useful in the preparation of the compounds of this invention.

### DETAILED DESCRIPTION OF THE INVENTION

It has now been discovered that substituted 1,2,3-triazoles of Formula (I) and Formula (II) are inhibitors of MetAP2. It has also now been discovered that selective inhibition of MetAP2 enzyme mechanisms by treatment with the inhibitors of Formula (I) and Formula (II), or a pharmaceutically acceptable salt thereof, represents a novel therapeutic and preventative approach to the treatment of a variety of disease states, including, but not limited to, cancer, haemangioma, proliferative retinopathy, rheumatoid arthritis, atherosclerotic neovascularization, psoriasis, ocular neovascularization and obesity.

The term “Ph” represents a phenyl ring. The terms “Het” or “heterocyclic” as used herein interchangeably at all occurrences, mean a stable heterocyclic ring, all of which are either saturated or unsaturated, and consist of carbon atoms and from one to three heteroatoms selected from the group consisting of N, O and S, and wherein the nitrogen may optionally be oxidized or quaternized. Ph and Het may be optionally unsubstituted or substituted as defined herein. Suitable “Het” include heterocycloalkyl groups, which are non-aromatic, monovalent monocyclic radicals, which are saturated or partially unsaturated, containing 5 to 8 ring atoms and 1 to 3 heteroatoms selected from nitrogen, oxygen and sulfur, including, but not limited to, pyrrolidyl, piperidyl, piperazinyl, morpholinyl, tetrahydro-2H-1,4-thiazinyl, tetrahydrofuryl, dihydrofuryl, tetrahydropyranyl, dihydropyranyl, 1,3-dioxolanyl, 1,3-dioxanyl, 1,4-dioxanyl, 1,3-oxathiolanyl, 1,3-oxathianyl. Suitable “Het” also include heteroaryl groups which are aromatic monovalent monocyclic, bicyclic, or tricyclic radicals, containing 5 to 8 ring atoms and 1 to 3 heteroatoms selected from nitrogen, oxygen and sulfur, including, but not limited to, thienyl, pyrrolyl, imidazolyl, pyrazolyl, furyl, isothiazolyl, furazanyl, isoxazolyl, thiazolyl, pyridyl, pyrazinyl, pyrimidinyl, pyridazinyl, triazinyl. Preferably, in this invention, suitable “Het” are monocyclic, heteroaryl groups, such as thienyl, furyl, thiazolyl, pyridyl, pyrazinyl or pyrimidinyl.

The term “C₁-C₆ alkyl” as used herein at all occurrences means a substituted and unsubstituted, straight or branched chain radical of 1 to 6 carbon atoms, unless the chain length is limited thereto (e.g., C₁-C₄ means a radical of 1 to 4 carbon atoms), including, but not limited to methyl, ethyl, n-propyl, isopropyl, n-butyl, isobutyl and t-butyl, pentyl, n-pentyl, isopentyl, neopentyl and hexyl and isomers thereof.

The term “C₁-C₆ alkoxy” is used herein at all occurrences to mean a straight or branched chain radical of 1 to 6 carbon atoms, unless the chain length is limited thereto (e.g. C₁-C₄ means a radical of 1 to 4 carbon atoms), bonded to an oxygen atom, including, but not limited to, methoxy, ethoxy, n-propoxy, isopropoxy, and the like.

In the substituents defined herein, the terms “alkyl” and “alkoxy” are also meant to include both monovalent and divalent straight or branched carbon chain radicals. For example, the term “(C₁-C₄ alkyl)HN—(C₁-C₄ alkyl)-” is meant to refer to a substituent having the bonding arrangement “(CH₃)HN—CH₂—” or “(CH₃)HN—CH₂(CH₃)CHCH₂—” and the term “HOC(O)—C₁-C₄ alkoxy-” is meant to refer to a substituent having the bonding arrangement: “HO—C(O)—CH₂—O—” or “HO—C(O)—(CH₃)CH—O—”.

The alkyl and alkoxy substituents/moieties as defined herein may be optionally unsubstituted or substituted. If substituents for an alkyl or alkoxy substituent/moiety are not specified, the alkyl or alkoxy substituent/moiety is intended to be unsubstituted.

The terms “hetero” or “heteroatom” as used herein interchangeably at all occurrences mean oxygen, nitrogen and sulfur.

The terms “halo” or “halogen” as used herein interchangeably at all occurrences mean F, Cl, Br, and I. The term “haloalkyl” as used herein means a straight or branched chain carbon radical that is substituted by one or more halogens (e.g., —CF₃, —CH₂CF₃, —CF₂CF₃, etc.).

The terms “hydroxy” or “hydroxyl” as used herein interchangeably is intended to mean the radical —OH. “Amino” is intended to mean the radical —NH₂.

Here and throughout this application the term “C₀” denotes the absence of the substituent group immediately following; for instance, in the moiety Ph-C₀-C₆ alkyl-, when C is 0, the substituent is phenyl.

It will be appreciated by those skilled in the art, that the compounds of this invention may exist in tautomeric forms, wherein the hydrogen on the triazole ring may exist on either N1 or N3, as shown below.

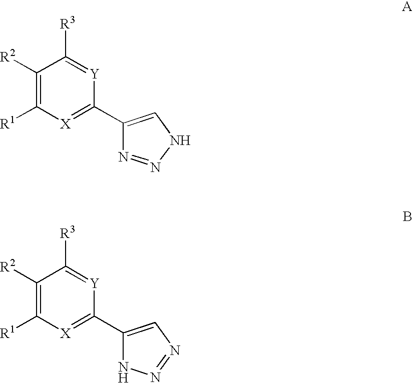

All tautomeric forms of the compounds described herein are intended to be encompassed within the scope of the present invention. As a convention, the compounds exemplified herein have been assigned names based on the structure of the tautomer of Formula A. It is to be understood that any reference to such named compounds is intended to encompass all tautomers thereof.

In the embodiments of this invention, R¹, R² and R³ are hydrogen, unless otherwise provided.

In the specific embodiments of this invention, both X and Y are CH, both X and Y are N, or only one of X or Y is —CR (e.g., when X is CR, Y is N or CH, or when Y is CR, X is N or CH).

In the specific embodiments of this invention, R¹ is selected from halogen, hydroxyl, Ph-C₀-C₆ alkoxy and C₁-C₆ alkyl. More specifically, R is Ph-O—, OH—, bromo or methyl.

In the specific embodiments of this invention, R¹ is selected from hydrogen, halogen, C₁-C₆ alkyl, C₁-C₆ alkoxy, Ph-C₀-C₆ alkoxy, C₁-C₆ haloalkyl, (C₁-C₄ alkoxyC(O)(C₁-C₄ alkyl)HN—, (Het-C₁-C₄ alkyl)HN—, wherein said Het is unsubstituted or substituted by one or more substituents independently selected from C₁-C₄ alkyl, hydroxy-C₁-C₄ alkyl- and hydroxyl, and (Ph-C₁-C₄ alkyl)HN—, wherein said Ph is substituted by one or more substituents independently selected from C₁-C₄ alkoxy, C₁-C₄ alkylC(O)O— and HOC(O)C₁-C₄ alkoxy. Specifically, R¹ is selected from the group hydrogen, chloro, bromo, iodo, Ph-O—, PhCH₂O—, MeO—, trifluoromethyl, methyl, ((2-(HOC(O)—CH₂O)Ph)—CH₂)HN, ((fur-2-yl)-CH₂)HN—, (2-methyl, 3-hydroxy, 5-hydroxymethyl-(pyrid-4-yl-CH₂₋)HN—, (2-HOC(O)—CH₂O-phenyl-CH₂—)HN—, (2-CH₃O-phenyl-CH₂—)HN—, (4-CH₃C(O)O-phenyl-CH₂—)HN—, and (CH₃OC(O)CH₂(H)N—. Preferably, R¹ is selected from hydrogen, halogen, C₁-C₆ alkyl, phenoxy, C₁-C₆ haloalkyl, (Het-C₁-C₄ alkyl)HN—, or (Ph-C₁-C₄ alkyl)HN—, wherein Ph is unsubstituted or substituted by C₁-C₄ alkoxy.

In the specific embodiments of this invention, R² is selected from hydrogen, halogen or C₁-C₆ alkyl. More specifically, R² is selected from hydrogen, fluoro, chloro, bromo, iodo or methyl.

In the specific embodiments of this invention, R³ is selected from hydrogen, halogen or C₁-C₆ alkyl. Specifically, R³ is selected from the group hydrogen, chloro, bromo, iodo and methyl.

In another specific embodiment of this invention, R¹ and R² or R² and R³, taken together with the atoms to which they are attached, form a 6-membered carbocyclic aromatic ring.

Preferred embodiments of this invention comprise compounds or methods of administering compounds according to Formula (I) or Formula (II), respectively, wherein when both X and Y are CH: R¹ is Ph-C₀-C₆ alkoxy, (Het-C₁-C₄ alkyl)HN—, or (Ph-C₁-C₄ alkyl)HN—, wherein Ph is unsubstituted or substituted by C₁-C₄ alkoxy; or R¹ and R² are each independently selected from halogen or C₁-C₆ alkyl, wherein when R¹ and R² are both halogen or are both C₁-C₆ alkyl, each of said halogen or C₁-C₆ alkyl is the same or different. Such preferred embodiments include compounds according to Formula (I) or Formula (II), wherein when both X and Y are CH: R¹ is Ph-C₀-C₁ alkoxy or (Het-C₁-C₂ alkyl)HN—; or R¹ and R² are each halogen, wherein each halogen is the same or different.

Preferred embodiments of the methods of this invention comprise administering compounds according to Formula (II) wherein when both X and Y are CH: R² is halogen or C₁-C₆ alkyl; or R¹ is halogen or C₁-C₆ alkyl.

Other preferred embodiments of this invention comprise compounds or methods of administering compounds according to Formula (I) and Formula (II), respectively, wherein when Y is CR and X is CH: R is Ph-C₀-C₆ alkoxy; or R is halogen and R¹ is selected from hydrogen, halogen or (Het-C₁-C₄ alkyl)HN—, R² is selected from hydrogen or halogen. Preferably, R¹ and R² are not both hydrogen when Y is CR, X is CH, R is halogen, R¹ is hydrogen, halogen or (Het-C₁-C₄ alkyl)HN— and R² is hydrogen or halogen.

Yet other preferred embodiments of this invention comprise compounds or methods of administering compounds according to Formula (I) and Formula (II), respectively, wherein when Y is N and X is CH: R¹ is C₁-C₆ alkyl or C₁-C₆ haloalkyl; or R² is halogen or C₁-C₆ alkyl; or R¹ and R³ are each C₁-C₆ alkyl; or R¹ and R², taken together with the atoms to which they are attached, form a 6-membered aromatic ring; or
when Y is N and X is CR: R is C₁-C₆ alkyl; or when Y is N: X is N.

Suitably, pharmaceutically acceptable salts of the compounds of Formula (I) or Formula (II) include, but are not limited to, salts with inorganic acids such as hydrochloride, sulfate, phosphate, diphosphate, hydrobromide, and nitrate, or salts with an organic acid such as malate, maleate, fumarate, tartrate, succinate, citrate, acetate, lactate, methanesulfonate, p-toluenesulfonate, palmitate, salicylate, and stearate.

The compounds of the present invention may contain one or more asymmetric carbon atoms and may exist in racemic and optically active forms. The stereocenters may be (R), (S) or any combination of R and S configuration, for example, (R,R), (R,S), (S,S) or (S,R). All of these compounds are within the scope of the present invention.

Novel intermediates that can be made according to the Schemes and Examples described herein and that are useful in making compounds of this invention are as follows: ethoxycarbonylmethyl-(3-ethynyl-phenyl)-amine, (2-methoxy-benzyl)-[3-ethynyl-phenyl]-amine, 5-hydroxymethyl-2-methyl-4-{[3-ethynyl-phenylamino]-methyl}-pyridin-3-ol, (4-acetoxy-3-methoxy-benzyl)-[3-ethynyl-phenyl]-amine, (2-carboxymethoxy-benzyl)-[3-ethynyl-phenyl]-amine, 1,2-dibromo-4-ethynylbenzene, 1-ethynyl-2-phenoxy-benzene, 3-(5-trimethylsilanyl-1H-1,2,3-triazol-4-yl)-isoquinoline, 2-ethynyl-4-trifluoromethyl-pyridine, 2-ethynyl-6-methoxy-pyridine, 2-ethynyl-4-ethyl-pyridine and 2-iodo4,5-dimethyl-pyridine, or a tautomer thereof, or a pharmaceutically active salt or solvate thereof.

Compounds of Formula (I) and Formula (II) include the following: ethoxycarbonylmethyl-(3-[1H-1,2,3-triazol-4-yl]phenyl)-amine, (2-methoxy-benzyl)-[3-(1H-1,2,3-trizol-4-yl)phenyl]-amine, 5-hydroxymethyl-2-methyl-4-{[3-(1H-1,2,3-triazol-4-yl)-phenylamino]-methyl}-pyridin-3-ol, (4-acetoxy-3-methoxy-benzyl)-[3-(1H-1,2,3-triazol-4-yl)-phenyl]-amine, (2-carboxymethoxy-benzyl)-[3-(1H-1,2,3-triazol-4-yl)-phenyl]-arnine, furan-2-ylmethyl-(4-bromo-3-[1H-1,2,3-triazol-4-yl]phenyl)amine, 2-(5-bromo-1H-1,2,3-triazol4-yl)-pyridine, 3-(1H-1,2,3-triazol-4-yl)-isoquinoline, 4-(3,4-dibromophenyl)-1H-1,2,3-triazole, 4-(3-bromophenyl)-1H-1,2,3-triazole, 4-(4-iodophenyl)-1H-1,2,3-triazole, 4-(3-phenoxy-phenyl)-1H-1,2,3-triazole, 4-(3-benzyloxy-phenyl)-1H-1,2,3-triazole, 4-(2-phenoxy-phenyl)-1H-1,2,3-triazole, 4-(2,4,5-tribromophenyl)-1H-1,2,3-triazole, 2-(1H-1,2,3-triazol-4-yl)-4-trifluoromethyl-pyridine, 4-ethyl-2-(1H-1,2,3-triazol-4-yl)-pyridine, 3-methyl-2-(1H-1,2,3-triazol-4-yl)-pyridine, 5-bromo-2-(1H-1,2,3-triazol-4-yl)-pyridine, 2-methoxy-6-(1H-1,2,3-triazol-4-yl)-pyridine, 2-(1H-1,2,3-triazol-4-yl)-pyrimidine, 4,5-dimethyl-2-(1H-1,2,3-triazol-4-yl)-pyridine, 2,4-dimethyl-6-(1H-1,2,3-triazol-4-yl)-pyridine and 4-(2-hydroxyphenyl)-1H-1,2,3-triazole.

Preferred compounds of Formula (I) and Formula (II) include the following: 3-(1H-1,2,3-triazol-4-yl)-isoquinoline, 4-(3,4-dibromophenyl)-1H-1,2,3-triazole, 4-(3-bromophenyl)-1H-1,2,3-triazole, 4-(4-iodophenyl)-1H-1,2,3-triazole, 4-(3-phenoxy-phenyl)-1H-1,2,3-triazole, 4-(2-phenoxy-phenyl)-1H-1,2,3-triazole, 2-(1H-1,2,3-triazol-4-yl)-4-trifluoromethyl-pyridine, 4-ethyl-2-(1H-1,2,3-triazol-4-yl)-pyridine, 3-methyl-2-(1H-1,2,3-triazol4-yl)-pyridine, 5-bromo-2-(1H-1,2,3-triazol-4-yl)-pyridine, 2-(1H-1,2,3-triazol-4-yl)-pyrimidine, and 2,4-dimethyl-6-(1H-1,2,3-triazol-4-yl)-pyridine.

General Methods

Compounds of Formulae (I) and (II) were prepared by methods analogous to those described in Scheme 1.

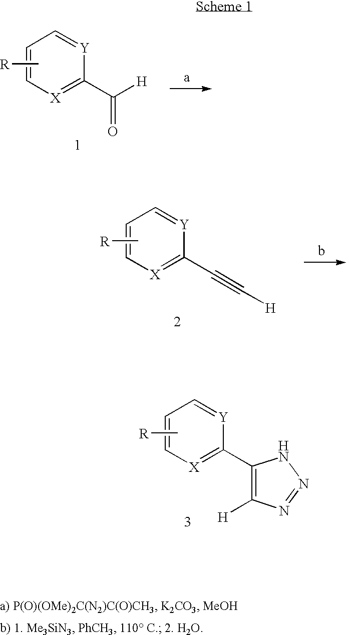

An aldehyde (such as 4-iodobenzaldehyde) (1-Scheme 1) was treated with 1-diazo-2-oxopropylphosphonate and potassium carbonate in dry methanol to provide 2-Scheme 1. Treatment of the acetylene (such as 4-iodo-1-ethynylbenzene, 2-Scheme 1) with azidotrimethylsilane in refluxing toluene, followed by addition of water afforded triazole 3-Scheme 1.

Compounds of Formulae (I) and (II), where R¹ is (Ph-C₁-C₄ alkyl)(C₁-C₄ alkyl)N—, (Ph-C₁-C₄ alkyl)HN—, (Het-C₁-C₄ alkyl)(C₁-C₄ alkyl)N—, (Het-C₁-C₄ alkyl)HN—, (C₁-C₄ alkyl)(C₁-C₄ alkyl)N— or (C₁-C₄ alkyl)HN—, exemplified in Scheme 2 where R¹ is Ph(CH₂)NH—, were prepared by methods analogous to those described as follows.

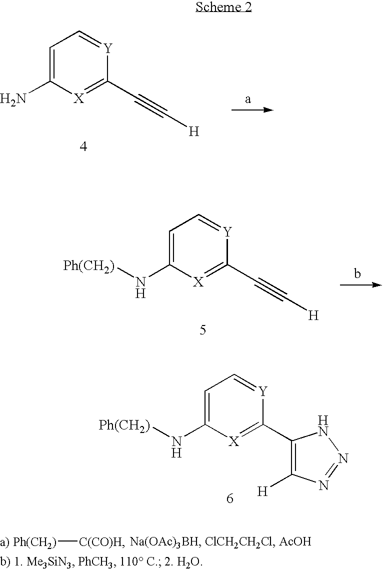

An alkynyl aniline (such as 3-ethynylphenylamine) was substituted by a reductive amination reaction with an aldehyde to provide 5-Scheme 2. Treatment of the acetylene (5-Scheme 2) with azidotrimethylsilane in refluxing toluene, followed by addition of water afforded 6-Scheme 2.

Compounds of Formulae (I) and (II), where X═N, were prepared by methods analogous to those described in Scheme 3.

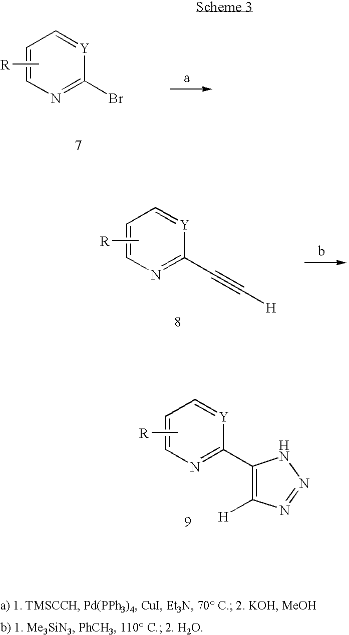

A bromo-pyridine (such as 2-bromo-4-trifluoromethyl-pyridine) was cross-coupled to a silylalkyne and the silyl group was removed by basic hydrolysis to provide 8-Scheme 3. Treatment of the acetylene (8-Scheme 3) with azidotrimethylsilane in refluxing toluene, followed by addition of water afforded 9-Scheme 3.

The pharmaceutically effective compounds of this invention (and the pharmaceutically acceptable salts thereof) are administered in conventional dosage forms prepared by combining a compound of this invention of Formula (I) or (II) (“active ingredient”) in an amount sufficient to treat cancer, haemangioma, proliferative retinopathy, rheumatoid arthritis, atherosclerotic neovascularization, psoriasis, ocular neovascularization or obesity (“MetAp2-mediated disease states”) with standard pharmaceutical carriers or diluents according to conventional procedures well known in the art. These procedures may involve mixing, granulating and compressing or dissolving the ingredients as appropriate to the desired preparation.

The pharmaceutical carrier employed may be, for example, either a solid or liquid. Exemplary of solid carriers is lactose, terra alba, sucrose, talc, gelatin, agar, pectin, acacia, magnesium stearate, stearic acid and the like. Exemplary of liquid carriers is syrup, peanut oil, olive oil, water and the like. Similarly, the carrier or diluent may include time delay material well known to the art, such as glyceryl monostearate or glyceryl distearate alone or with a wax.

A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg to about 1000 mg. When a liquid carrier is used, the preparation will be in the form of a syrup, emulsion, soft gelatin capsule, sterile injectable liquid such as an ampule or nonaqueous liquid suspension.

The active ingredient may also be administered topically to a mammal in need of treatment or prophylaxis of MetAP2-mediated disease states. The amount of active ingredient required for therapeutic effect on topical administration will, of course, vary with the compound chosen, the nature and severity of the disease state being treated and the mammal undergoing treatment, and is ultimately at the discretion of the physician. A suitable dose of an active ingredient is 1.5 mg to 500 mg for topical administration, the most preferred dosage being 1 mg to 100 mg, for example 5 to 25 mg administered two or three times daily.

By topical administration is meant non-systemic administration and includes the application of the active ingredient externally to the epidermis, to the buccal cavity and instillation of such a compound into the ear, eye and nose, and where the compound does not significantly enter the blood stream. By systemic administration is meant oral, intravenous, intraperitoneal and intramuscular administration.

While it is possible for an active ingredient to be administered alone as the raw chemical, it is preferable to present it as a pharmaceutical formulation. The active ingredient may comprise, for topical administration, from 0.001% to 10% w/w, e.g. from 1% to 2% by weight of the formulation although it may comprise as much as 10% w/w but preferably not in excess of 5% w/w and more preferably from 0.1% to 1% w/w of the formulation.

The topical formulations of the present invention, both for veterinary and for human medical use, comprise an active ingredient together with one or more acceptable carrier(s) therefor and optionally any other therapeutic ingredient(s). The carrier(s) must be “acceptable” in the sense of being compatible with the other ingredients of the formulation and not deleterious to the recipient thereof.

Formulations suitable for topical administration include liquid or semi-liquid preparations suitable for penetration through the skin to the site of inflammation such as liniments, lotions, creams, ointments or pastes, and drops suitable for administration to the eye, ear or nose.

Drops according to the present invention may comprise sterile aqueous or oily solutions or suspensions and may be prepared by dissolving the active ingredient in a suitable aqueous or alcoholic solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol. The formulation may incorporate any suitable surface-active agent such as an anionic, cationic or non-ionic surfactant such as esters or polyoxyethylene derivatives thereof. Suspending agents such as natural gums, cellulose derivatives or inorganic materials such as silicaceous silicas, and other ingredients such as lanolin, may also be included.

The active ingredient may also be administered by inhalation. By “inhalation” is meant intranasal and oral inhalation administration. Appropriate dosage forms for such administration, such as an aerosol formulation or a metered dose inhaler, may be prepared by conventional techniques. The daily dosage amount of the active ingredient administered by inhalation is from about 0.1 mg to about 100 mg per day, preferably about 1 mg to about 10 mg per day.

In one aspect, this invention relates to a method of treating cancer, haemangioma, proliferative retinopathy, rheumatoid arthritis, atherosclerotic neovascularization, psoriasis, ocular neovascularization or obesity, all in mammals, preferably humans, which comprises administering to such mammal an effective amount of a MetAP2 inhibitor, in particular, a compound of this invention.

By the term “treating” is meant either prophylactic or therapeutic therapy. Such compound can be administered to such mammal in a conventional dosage form prepared by combining the compound of this invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. The compound is administered to a mammal in need of treatment for cancer, haemangioma, proliferative retinopathy, rheumatoid arthritis, atherosclerotic neovascularization, psoriasis, ocular neovascularization or obesity, in an amount sufficient to decrease symptoms associated with these disease states. The route of administration may be oral or parenteral.

The term parenteral as used herein includes intravenous, intramuscular, subcutaneous, intra-rectal, intravaginal or intraperitoneal administration. The subcutaneous and intramuscular forms of parenteral administration are generally preferred. The daily parenteral dosage regimen will preferably be from about 30 mg to about 300 mg per day of active ingredient. The daily oral dosage regimen will preferably be from about 100 mg to about 2000 mg per day of active ingredient.

It will be recognized by one of skill in the art that the optimal quantity and spacing of individual dosages of a compound of this invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of the compound given per day for a defined number of days, can be ascertained by those skilled in the art using conventional course of treatment determination tests.

### EXAMPLES

The invention will now be described by reference to the following examples, which are merely illustrative and are not to be construed as a limitation of the scope of the present invention. In the Examples, proton NMR spectra were performed upon a Bruker 400 MHz NMR spectrometer, unless otherwise indicated.

### Example 1

### Preparation of ethoxycarbonylmethyl-(3-[1H-1,2,3-triazol-4-yl]phenyl)-amine

### a) ethoxycarbonylmethyl-(3-ethynyl-phenyl)-amine

To a stirring solution of 3-ethynylphenylamine (0.59 g, 5.0 mmol) and glyoxylic acid ethyl ester (1.02 g, 5.0 mmol) in 1,2-dichloroethane (15 ml) was added acetic acid (0.29 ml, 5.0 mmol) and sodium triacetoxyborohydride (1.6 g, 7.5 mmol). After stirring at room temperature for 16 h, aqueous sodium bicarbonate (saturated) and diethyl ether were added. The organic layer was washed with additional sodium bicarbonate, dried (MgSO₄) and evaporated. Purification via silica gel chromatography gave the title compound as an oil (80% yield). MS (ESI) 204.2 (M+H)⁺.

### b) ethoxycarbonylmethyl-(3-[1H-1,2,3-triazol-4-yl]phenyl)-amine

To a stirring solution of ethoxycarbonylmethyl-(3-ethynyl-phenyl)-amine (0.81 g, 3.0 mmol) in toluene (4 ml) under argon was added trimethylsilylazide (1 ml, 8 mmol). The resulting solution was heated to reflux for 3 days. To this mixture was added water (1 ml) and after evaporation, the resulting residue was purified by preparative HPLC to afford the title compound as a white solid (0.04 g, 3%). ¹H-NMR (400 MHz, CD₃OD): δ 8.07 (s, 1H), 7.22 (dt, J=7.9, 1.9 Hz, 1H), 7.09-7.14 (m, 2H), 6.64 (dd, J=8.1, 1.4 Hz, 1H), 4.23 (q, J=7.1 Hz, 2H), 3.99 (s, 2H), 1.28 (t, J=7.2 Hz, 3H). MS (ESI) 247.0 (M+H)⁺.

### Example 2

### Preparation of (2-methoxy-benzyl)-[3-(1H-1,2,3-trizol-4-yl)phenyl]-amine

### a) (2-methoxy-benzyl)-[3-ethynyl-phenyl]-amine

Following the procedure of Example 1a, except substituting o-anisaldehyde for glyoxylic acid ethyl ester, the title compound was obtained as an oil. MS (ESI) 238.0 (M+H)⁺.

### b) (2-methoxy-benzyl)-[3-(1H-1,2,3-trizol-4-yl)phenyl]-amine

Following the procedure of Example 1b, except substituting (2-methoxy-benzyl)-[3-ethynyl-phenyl]-amine for ethoxycarbonylmethyl-(3-[1H-1,2,3-triazol4-yl]phenyl)-amine, the title compound was prepared as a white solid (2% yield over two steps). ¹H-NMR (400 MHz, CD₃OD): δ 8.01 (s, 1H), 6.86-7.33 (m, 7H), 6.65 (dd, J=10.29, 2.22 Hz, 1H), 4.36 (s, 2H), 3.89 (s, 3H). MS (ESI) 281.2 (M+H)⁺.

### Example 3

### Preparation of 5-hydroxymethyl-2-methyl-4-{[3-(1H-1,2,3-triazol-4-yl)-phenylamino]-methyl}-pyridin-3-ol

### a) 5-hydroxymethyl-2-methyl4-{[3-ethynyl-phenylamino]-methyl}-pyridin-3-ol

Following the procedure of Example 1a, except substituting pyridoxal hydrochloride for glyoxylic acid ethyl ester, the title compound was obtained as a tan solid (57% yield). MS (ESI) 269.0 (M+H)⁺.

### b) 5-hydroxymethyl-2-methyl4-{[3-(1H-1,2,3-triazol-4-yl)-phenylamino]-methyl}-pyridin-3-ol

Following the procedure of Example 1b, except substituting 5-hydroxymethyl-2-methyl-4-{[3-ethynyl-phenylamino]-methyl}-pyridin-3-ol for ethoxycarbonylmethyl-(3-[1H-1,2,3-triazol-4-yl]phenyl)-amine, the title compound was prepared as an oil (1% yield). ¹H-NMR (400 MHz, CD₃OD): δ 8.07 (s, 1H), 7.92 (s, 1H), 7.25-7.30 (m, 3H), 6.83 (d, J=3.4 Hz, 1H), 4.73 (s, 2H), 4.58 (s, 2H), 2.43 (s, 3H). MS (ESI) 312.2 (M+H)⁺.

### Example 4

### Preparation of (4-acetoxy-3-methoxy-benzyl)-[3-(1H-1,2,3-triazol-4-yl)-phenyl]-amine

### a) (4-acetoxy-3-methoxy-benzyl)-[3-ethynyl-phenyl]-amine

Following the procedure of Example 1a, except substituting 4-acetoxy-5-methoxybenzaldehyde for glyoxylic acid ethyl ester, the title compound was obtained as an oil (89% yield). MS (ESI) 296.0 (M+H)⁺.

### b) (4-acetoxy-3-methoxy-benzyl)-[3-(1H-1,2,3-triazol-4-yl)-phenyl]-amine

Following the procedure of Example 1b, except substituting (4-acetoxy-3-methoxy-benzyl)-[3-ethynyl-phenyl]-amine for ethoxycarbonylmethyl-(3-[1H-1,2,3-triazol-4-yl]phenyl)-amine, the title compound was prepared as an oil (7% yield). ¹H-NMR (400 MHz, CD₃OD): δ 8.05 (s, 1H), 6.63-7.33 (m, 7H), 4.36 (s, 2H), 3.78 (s, 3H), 2.25 (s, 3H). MS (ESI) 339.2 (M+H)⁺.

### Example 5

### Preparation of (2-carboxymethoxy-benzyl)-[3-(1H-1,2,3-triazol-4-yl)-phenyl]-amine

### a) (2-carboxymethoxy-benzyl)-[3-ethynyl-phenyl]-amine

Following the procedure of Example 1a, except substituting 2-formyl-phenoxyacetic acid for glyoxylic acid ethyl ester, the title compound was obtained as an oil (90% yield). MS (ESI) 282.0 (M+H)⁺.

### b) (2-carboxymethoxy-benzyl)-[3-(1H-1,2,3-triazol-4-yl)-phenyl]-amine

Following the procedure of Example 1b, except substituting (2-carboxymethoxy-benzyl)-[3-ethynyl-phenyl]-amine for ethoxycarbonylmethyl-(3-[1H-1,2,3-triazol-4-yl]phenyl)-amine, the title compound was prepared as a white solid (6% yield). ¹H-NMR (400 MHz, CD₃OD): δ 8.04 (s, 1H), 6.72-7.34 (m, 8H), 4.55 (s, 2H), 4.42 (s, 2H). MS (ESI) 325.2 (M+H)⁺.

### Example 6

### Preparation of furan-2-ylmethyl-(4-bromo-3-[1H-1,2,3-triazol-4-yl]phenyl)amine

### a) furan-2-ylmethyl-(3-[1H-1,2,3-triazol-4-yl]phenyl)amine

Prepared according to the procedure described in International Patent Application No. PCT/US01/11979 or by following the procedure of Example 1, above, except substituting furfural for glyoxylic acid ethyl ester in step a, the title compound was prepared as a white solid (14% yield). ¹H-NMR (400 MHz, CD₃OD): δ 8.06 (s, 1H), 7.43 (d, J=1.0 Hz, 1H), 7.09-7.22 (m, 3H), 6.72 (d, J=8.1 Hz, 1H), 6.34-6.35 (m, 1H), 6.28 (d, J=3.2 Hz, 1H), 4.36 (s, 2H). MS (ESI) 241.2 (M+H)⁺.

### b) furan-2-ylmethyl-(4-bromo-3-[1H-1,2,3-trizaol4-yl]phenyl)amine

To furan-2-ylmethyl-(3-[1H-1,2,3-triazol4-yl]phenyl)amine (56 mg, 0.23 mmol) in acetic acid (0.5 ml) was added bromine (12 uL, 0.23 mmol). After 30 min of stirring at room temperature (RT), water (10 ml) and ethyl acetate (10 ml) were added. The aqueous layer was neutralized with saturated NaHCO₃. The water layer was washed with ethyl acetate three times and the collected organic layers were dried, filtered, and evaporated. The resulting residue was purified by preparative HPLC to afford the title compound as a yellow oil (23% yield). ¹H-NMR (400 MHz, CD₃OD): δ 8.18 (s, 1H), 7.38-7.42 (m, 2H), 7.06 (d, J=2.7 Hz, 1H), 6.63-6.67 (dd, J=8.8, 2.9 Hz, 1H), 6.33 (s, 1H), 6.27 (d, J=3.0 Hz, 1H), 4.32 (s, 2H). MS (ESI) 319.0 (M+H)⁺.

### Example 7

### Preparation of 4-(3-bromophenyl)-1H-1,2,3-triazole

### a) 1-bromo-3-ethynylbenzene

To a stirring solution of 3-bromobenzaldehyde (0.74 g, 4.0 mmol) in dry methanol (20 ml) was added potassium carbonate (1.1 g, 8 mmol) and 1-diazo-2-oxopropylphosphonate (0.96 g, 5.0 mmol, Calant, P.; D'Haenens, L.; Vandewalle, M. Synth. Commun. 1984, 14, 155). After 4 h of stirring at room temperature, aqueous sodium bicarbonate (5%, 50 ml) and hexanes (50 ml) were added. The organic layer was collected, dried (MgSO₄) and filtered through a short silica plug. Evaporation yielded the title compound as an oil (84% yield).

### b) 4-(3-bromophenyl)-1H-1,2,3-triazole

Following the procedure of Example 1b, except substituting 1-bromo-3-ethynylbenzene for ethoxycarbonylmethyl-(3-ethynyl-phenyl)-amine, the title compound was prepared as a white solid (3% yield). ¹H-NMR (400 MHz, CD₃O D): δ 8.19 (s, 1H), 8.05 (s, 1H), 7.83 (d, J=7.8 Hz, 1H), 7.52 (d, J=8.0 Hz, 1H), 7.37 (t, J=7.9 Hz, 1H). MS (ESI) 224.0 (M+H)⁺.

### Example 8

### Preparation of 4-(4-iodophenyl)-1H-1,2,3-triazole

### a) 4-iodo-1-ethynylbenzene

Following the procedure of Example 7a, except substituting 4-iodobenzaldehyde for 3-bromobenzaldehyde, the title compound was obtained as a white solid (71% yield).

### b) 4-(4-iodophenyl)-1H-1,2,3-triazole

Following the procedure of Example 1b, except substituting 4-iodo-1-ethynylbenzene for ethoxycarbonylmethyl-(3-ethynyl-phenyl)-amine, the title compound was prepared as a white solid (15% yield). ¹H-NMR (400 MHz, CD₃OD): δ 8.19 (s, 1H), 7.82 (d, J=8.4 Hz, 2H), 7.64 (d, J=8.4 Hz, 2H). MS (ESI) 271.8 (M+H)⁺.

### Example 9

### Preparation of 4-(3,4-dibromophenyl)-1H-1,2,3-triazole and 4-(2,4,5-tribromophenyl)-1H-1,2,3-triazole

### a) 1,2-dibromo-4-ethynylbenzene

Following the procedure of Example 7a, except substituting 3,4-dibromobenzaldehyde for 3-bromobenzaldehyde, the title compound was obtained as a white solid (81% yield).

### b) 4-(3,4-dibromophenyl)-1H-1,2,3-triazole and 4-(2,4,5-tribromophenyl)-1H-1,2,3-triazole

Following the procedure of Example 1b, except 1,2-dibromo-4-ethynylbenzene for ethoxycarbonylmethyl-(3-ethynyl-phenyl)-amine, compound 4-(3,4-dibromophenyl)-1H-1,2,3-triazole was obtained as the major product (33% yield). ¹H-NMR (400 MHz, CD₃OD): δ 8.25 (s, 1H), 8.22 (s, 1H), 7.76 (s, 2H). MS (ESI) 303.8 (M+H)⁺. Purification by preparative HPLC also afforded 4-(2,4,5-tribromophenyl)-1H-1,2,3-triazole as a minor product (0.4% yield). ¹H-NMR (400 MHz, CD₃OD): δ 8.30 (s, 1H), 8.20 (s, 2H). MS (ESI) 381.6 (M+H)⁺.

### Example 10

### Preparation of 4-(3-phenoxy-phenyl)-1H-1,2,3-triazole

### a) 1-ethynyl-3-phenoxy-benzene

Following the procedure of Example 7a, except substituting 3-phenoxybenzaldehyde for 3-bromobenzaldehyde, the title compound was obtained as an oil (89% yield).

### b) 4-(3-phenoxy-phenyl)-1H-1,2,3-triazole

Following the procedure of Example 1b, except substituting 1-ethynyl-3-phenoxy-phenyl for ethoxycarbonylmethyl-(3-ethynyl-phenyl)-amine, the title compound was prepared as a white solid (18% yield). ¹H-NMR (400 MHz, CD₃OD): δ 8.16 (s, 1H), 7.38-7.61 (m, 5H), 6.98-7.18 (m, 4H). MS (ESI) 238.0 (M+H)⁺.

### Example 11

### Preparation of 4-(3-benzyloxy-phenyl)-1H-1,2,3-triazole

### a) 1-ethynyl-3-benzyloxy-benzene

Following the procedure of Example 7a, except substituting 3-benzyloxybenzaldehyde for 3-bromobenzaldehyde, the title compound was obtained as an oil (52% yield).

### b) 4-(3-benzyloxy-phenyl)-1H-1,2,3-triazole

Following the procedure of Example 1b, except substituting 1-ethynyl-3-benzyloxy-benzene for ethoxycarbonylmethyl-(3-ethynyl-phenyl)-amine, the title compound was prepared as a white solid (11% yield). ¹H-NMR (400 MHz, CD₃OD): δ 8.16 (s, 1H), 7.31-7.49 (m, 8H), 7.02 (d, J=8.1 Hz, 1H), 5.16 (s, 2H). MS (ESI) 252.0 (M+H)⁺.

### Example 12

### Preparation of 4-(2-phenoxy-phenyl)-1H-1,2,3-triazole

### a) 1-ethynyl-2-phenoxy-benzene

Following the procedure of Example 7a, except substituting 2-phenoxybenzaldehyde for 3-bromobenzaldehyde, the title compound was obtained as a white solid (62% yield).

### b) 4-(2-phenoxy-phenyl)-1H-1,2,3-triazole

Following the procedure of Example 1b, except substituting 1-ethynyl-2-phenoxy-benzene for ethoxycarbonylmethyl-(3-ethynyl-phenyl)-amine, the title compound was prepared as a white solid (10% yield). ¹H-NMR (400 MHz, CD₃OD): δ CD₃OD δ 8.08-8.12 (m, 2H), 7.33-7.37 (m, 3H) 7.35 (t, J=7.8 Hz, 1H), 7.11 (t, J=7.4 Hz, 1H), 6.97-6.99 (m, 3 H). MS (ESI) 238.0 (M+H)⁺.

### Example 13

### Preparation of 2-(1H-1,2,3-triazol-4-yl)-phenol

### a) 1-ethynyl-2-benzyloxy-benzene

Prepared according to the procedure described in International Patent Application No. PCT/US01/11979 or by following the procedure of Example 7a, above, except substituting 2-benzyloxybenzaldehyde for 3-bromobenzaldehyde, the title compound was obtained as an oil (90% yield).

### b) 4-(2-benzyloxy-phenyl)-1H-1,2,3-triazole

Following the procedure of Example 1b, except substituting 1-ethynyl-2-benzyloxy-benzene for ethoxycarbonylmethyl-(3-ethynyl-phenyl)-amine, the title compound was prepared as an oil (28% yield). MS (ESI) 252.2 (M+H)⁺.

### c) 2-(1H-1,2,3-triazol4-yl)-phenol

A solution of 4-(2-benzyloxy-phenyl)-1H-1,2,3-triazole (30 mg, 0.12 mmol) in ethyl acetate:methanol (6 ml, 2:1) was purged by argon and then was added palladium on carbon (10 w.t. %) and a hydrogen balloon. After two hours stirring, the reaction mixture was filtered with ethyl acetate. After removing solvents, the residue was purified by preparative HPLC to give the title compound as a white solid (50% yield). ¹H-NMR (400 MHz, CD₃OD): δ 8.25 (s, 1H), 7.82 (d, J=7.6 Hz, 1H), 7.22 (t, J=7.6 Hz, 1H), 6.97-6.92 (m, 2H). MS (ESI) 162.0 (M+H)⁺.

### Example 14

### Preparation of 3-(1H-1,2,3-triazol-4-yl)-isoquinoline

### a) 3-(5-trimethylsilanyl-1H-1,2,3-triazol-4-yl)-isoquinoline

To a stirring solution of trimethylsilyl diazomethane (0.7 uL, 2.0 M in hexanes, 1.3 mmol) in THF (15 ml) at 0 C under argon was added dropwise n-BuLi (0.8 uL, 1.6 M in heaxanes, 0.3 mmol) and stirring was continued for 20 min when 3-isoquinolinecarbonitrile (190 mg, 1.2 mmol) in THF (5 ml) was added. Stirring was continued at 0° C. for 3 hours then aqueous ammonium chloride was added. The organic product was extracted into ethyl acetate, which was dried and evaporated. Silica gel purification (20% ethyl acetate:80% hexanes ) gave the title compound as a tan oil (241 mg, 73% yield). MS (ESI) 269.0 (M+H)⁺.

### b) 3-(1H-1,2,3-triazol4-yl)-isoquinoline

To a stirring solution of 3-(5-trimethylsilanyl-1H-1,2,3-triazol4-yl)-isoquinoline (0.19 g, 0.7 mmol) in ethanol (1.5 ml) was added potassium hydroxide (2 M, 3 ml) and the mixture was heated to reflux for 1 h. After cooling, the reaction was neutralized with hydrochloric acid (1M) and the organics were extracted into ethyl acetate. The product was isolated from both the organic and aqueous layer by preparative HPLC to give the title compound as a tan solid (24 mg, 18% yield). ¹H-NMR (400 MHz, CD₃OD): δ 9.30 (s, ₁H), 8.37-8.41 (m, 2H), 8.12 (d, J=8.1 Hz, 1H), 8.00 (d, J=8.2 Hz, 1H), 7.81 (t, J=7.5 Hz, 1H), 7.70 (t, J=7.4 Hz, 1H). MS (ESI) 197.0 (M+H)⁺.

### Example 15

### Preparation of 2-(1H-1,2,3-triazol-4-yl)-4-trifluoromethyl-pyridine

### a) 2-ethynyl4-trifluoromethyl-pyridine

To a stirring solution of 2-bromo-4-trifluoromethyl-pyridine (0.45 g, 2.0 mmol) in triethylamine (3 ml) under argon was added copper iodide (0.09 g, 0.4 mmol), tetrakis(triphenylphosphine)palladium (0.26 g, 0.2 mmol), and trimethylsilyl acetylene (0.62 ml, 4.4 mmol). The reaction was heated to 70° C. for 2 h, the cooled and evaporated. The residue was purified by silica gel chromatography (5% ethyl acetate:95% hexanes) to give 0.15 g of 2-trinethylsilanylethynyl-4-trifluoromethyl-pyridine. This intermediate was dissolved in methanol (0.5 ml) and potassium hydroxide (1 M aqueous solution, 2 ml) was added. The mixture was stirred at RT for 1 h then the base was neutralized with 3N hydrochloric acid. The product was collected by extraction into diethyl ether, which was then dried, evaporated, and filtered through a short silica gel plug to give the title compound as an oil (32% yield).

### b) 2-(1H-1,2,3-triazol-4-yl)4-trifluoromethyl-pyridine

Following the procedure of Example 1b, except substituting 2-ethynyl-4-trifluoromethyl-pyridine for ethoxycarbonylmethyl-(3-ethynyl-phenyl)-amine, the title compound was prepared as a tan solid (45% yield). ¹H-NMR (400 MHz, CD₃OD): δ 8.87 (d, J=5.1 Hz, 1H), 8.42 (s, 1H), 8.31 (s, 1h), 7.66 (d, J=5.2 Hz, 1H). MS (ESI) 215.0 (M+H)⁺.

### Example 16

### Preparation of 3-methyl-2-(1H-1,2,3-triazol-4-yl)-pyridine

### a) 2-ethynyl-3-methyl-pyridine

Following the procedure of Example 15a, except substituting 2-bromo-3-methyl-pyridine for 2-bromo-4-trifluoromethyl-pyridine, the title compound was obtained as an oil. (58% yield). MS (ESI) 117.0 (M+H)⁺.

### b) 3-methyl-2-(1H-1,2,3-triazol-4-yl)-pyridine

Following the procedure of Example 1b, except substituting 2-ethynyl-3-methyl-pyridine for ethoxycarbonylmethyl-(3-ethynyl-phenyl)-amine, the title compound was prepared as a white solid (35% yield). ¹H-NMR (400 MHz, CD₃OD): δ 8.48 (d, J=4.0 Hz, 1H), 8.20 (s, 1H), 7.79 (d, J=8.0 Hz, 1H), 7.35 (m, 1H), 3.32 (s, 3H). MS (ESI) 161.0 (M+H)⁺.

### Example 17

### Preparation of 5-bromo-2-(1H-1,2,3-triazol-4-yl)-pyridine

### a) 5-bromo-2-ethynyl-pyridine

Following the procedure of Example 15a, except substituting 2,5-dibromo-pyridine for 2-bromo-4-trifluoromethyl-pyridine, the title compound was obtained as an oil. (30% yield). MS (ESI) 182.0 (M+H)⁺.

### b) 5-bromo-2-(1H-1,2,3-triazol-4-yl)-pyridine

Following the procedure of Example 1b, except substituting 5-bromo-2-ethynyl-pyridine for ethoxycarbonylmethyl-(3-ethynyl-phenyl)-amine, the title compound was prepared as a white solid. (34% yield). ¹H-NMR (400 MHz, CD₃OD): δ 8.70 (d, J=2.0 Hz, 1H), 8.30 (s, 1H), 8.10 (d, J=2.4 Hz, 1H), 7.99 (t, J=6.51 Hz, 1H). MS (ESI) 226.0 (M+H)⁺.

### Example 18

### Preparation of 2-methoxy-6-(1H-1,2,3-triazol-4-yl)-pyridine

### a) 2-ethynyl-6-methoxy-pyridine

Following the procedure of Example 15a, except substituting 2-bromo-6-methoxy-pyridine for 2-bromo4-trifluoromethyl-pyridine, the title compound was obtained as an oil. (70% yield). ¹H-NMR (400 MHz, CD₃OD): δ 7.34 (t, J=6.0 Hz, 1H), 6.90 (d, J=8.0 Hz, 1H), 6.55 (d, J=8.0 Hz), 3.76 (s, 3H), 2.93 (s, 1H).

### b) 2-methoxy-6-(1H-1,2,3-triazol-4-yl)-pyridine

Following the procedure of Example 1b, except substituting 2-ethynyl-6-methoxy-pyridine for ethoxycarbonylmethyl-(3-ethynyl-phenyl)-amine, the title compound was prepared as a white solid. (40% yield). ¹H-NMR (400 MHz, CD₃OD): δ 8.28 (s, 1H), 7.74 (t, J=7.8 Hz, 1H), 7.57 (d, J=4 Hz, 1H), 6.76 (d, J=8.0 Hz, 1H), 4.01 (s, 3H). MS (ESI) 177.2 (M+H)⁺.

### Example 19

### Preparation of 4-ethyl-2-(1H-1,2,3-triazol4-yl)-pyridine

### a) 2-ethynyl-4-ethyl-pyridine

Following the procedure of Example 15a, except substituting 2-bromo-4-ethyl-pyridine for 2-bromo4-trifluoromethyl-pyridine, the title compound was obtained as an oil. (53% yield). MS (ESI) 132.0 (M+H)⁺.

### b) 4-ethyl-2-(1H-1,2,3-triazol-4-yl)-pyridine

Following the procedure of Example 1b, except substituting 2-ethynyl4-ethyl-pyridine for ethoxycarbonylmethyl-(3-ethynyl-phenyl)-amine, the title compound was prepared as a white solid. (40% yield). ¹H-NMR (400 MHz, CD₃OD): δ 8.48 (d, J=4.0 Hz, 1H), 8.31 (s, 1H), 7.94(s, 1H), 7.27 (d, J=4.0 Hz, 1H), 2.78 (q, j=4.0 Hz, 2H), 1.34 (t, J=6.0 Hz, 3H). MS (ESI) 175.2 (M+H)⁺.

### Example 20

### Preparation of 2-(1H-1,2,3-triazol-4-yl)-pyrimidine

### a) 2-ethynyl-pyrimidine

Following the procedure of Example 15a, except substituting 2-bromo-pyrimidine for 2-bromo-4-trifluoromethyl-pyridine, the title compound was obtained as an oil. (54% yield). MS (ESI) 105.0 (M+H)⁺.

### b) 2-(1H-1,2,3-triazol-4-yl)-pyrimidine

Following the procedure of Example 1b, except substituting 2-ethynyl-pyrimidine for ethoxycarbonylmethyl-(3-ethynyl-phenyl)-amine, the title compound was prepared as a white solid. (30% yield). ¹H-NMR (400 MHz, CD₃OD): δ 8.88 (d, J=4.0 Hz, 2H), 8.44 (s, 1H), 7.44 (t, J=4.0,1H). MS (ESI) 147.0 (M+H)⁺.

### Example 21

### Preparation of 4,5-dimethyl-2-(1H-1,2,3-triazol-4-yl)-pyridine

### a) 2-iodo-4,5-dimethyl-pyridine

To a solution of 2-dimethylaminoethanol (2.8 ml, 33 mmol) in hexane (15 ml) cooled at 0° C. was added dropwise n-BuLi (34.9 ml, 40 mmol). After 15 min, a solution of 3,4-lutidine (1 g, 9.4 mmol) in hexane (10 ml) was added and the orange solution stirred for 1 hr at 0° C. After cooling to −78° C., a solution of I₂ in THF (20 ml) was added. The reaction mixture was maintained at −78° C. for 1 hr and then allowed to warm to room temperature. Hydrolysis at 0° C. with water was followed by extraction with diethyl ether and drying over MgSO₄. After evaporation of solvent, the crude product was purified by silica gel chromatography (hexanes:ethyl acetate, 3:1) to give the title compound as a yellow oil (46% yield). MS (ESI) 234.0 (M+H)⁺.

### b) 2-ethynyl-4,5-dimethyl-pyridine

Following the procedure of Example 15a, except substituting 2-iodo-4,5-dimethyl-pyridine for 2-bromo4-trifluoromethyl-pyridine, the title compound was obtained as an oil. (24% yield). MS (ESI) 132.0 (M+H)⁺.

### c) 4,5-dimethyl-2-(1H-1,2,3-triazol-4-yl)-pyridine

Following the procedure of Example 1b, except substituting 2-ethynyl-4,5-dimethyl-pyridine for ethoxycarbonylmethyl-(3-ethynyl-phenyl)-amine, the title compound was prepared as a white solid. (30% yield). ¹H-NMR (400 MHz, CD₃OD): δ 8.32 (s, 1H), 8.25 (s, 1H), 7.85( s, 1H), 2.41 (s, 3H), 2.34(s, 3H). MS (ESI) 175.0 (M+H)⁺.

### Example 22

### Preparation of 2,4-dimethyl-6-(1H-1,2,3-triazol4-yl)-pyridine

### a) 2,4-dimethyl-6-bromo-pyridine

To hydroboromic acid (47%, 14 ml) in a flask was added 2-amino-4,6-dimethyl pyridine (2.5 g, 20 mmol) followed by bromine (4 ml) at 0° C. Sodium nitrite (4.6 g) in water (7 ml) was then added dropwise while the temperature was kept below 0° C. After 30 min, a solution of sodium hydroxide (10 g) in water (40 ml) was added. The reaction mixture was extracted with ether and the combined organic layers were dried over Na₂SO₄. After the solvents was removed the crude mixture was purified by silica gel chromatography (hexane:ethyl acetate, 3:1) to give the title compound as a yellow oil (50% yield). MS (ESI) 188.0 (M+H)⁺.

### b) 2,4-dimethyl-6-ethynyl-pyridine

Following the procedure of Example 15a, except substituting 2-bromo-4,6-dimethyl-pyridine for 2-bromo-4-trifluoromethyl-pyridine, the title compound was obtained as an oil. (55% yield).

### c) 2,4-dimethyl-6-(1H-1,2,3-triazol-4-yl)-pyridine

Following the procedure of Example 1b, except substituting 2,4-dimethyl-6-ethynyl-pyridine for ethoxycarbonylmethyl-(3-ethynyl-phenyl)-amine, the title compound was prepared as a white solid. (40% yield). ¹H-NMR (400 MHz, CD₃OD): δ 8.31 (s, 1H), 7.69 (s, 1H), 7.12 (s, 1H), 2.55 (s, 3H), 2.41 (s, 3H). MS (ESI) 175.2 (M+H)⁺.

### Biological Evaluation

Direct Spectrophotometric Assays of hMetAP2:

The hMetAP2 activity can be measured by direct spectrophotometric assay methods using alternative substrates, L-methionine-p-nitroanilide (Met-pNA) and L-methionine-7-amido4-methylcoumarin (Met-AMC). The formation of p-nitroaniline (pNA) or 7-amido-4-methylcoumarin (AMC) was continuously monitored by increasing absorbance or fluorescence at 405 nm and 460 nm, respectively, on a corresponding plate reader. All assays were carried out at 30° C. The fluorescence or spectrophotometric plate reader was calibrated using authentic pNA and AMC from Sigma, respectively. For a typical 96-well plate assay, the increase in the absorbance (at 405 nm for pNA) or the fluorescence emission (λₑₓ=360 nm, λₑₘ=460 nm, for AMC) of a 50 μL assay solution in each well was used to calculate the initial velocity of hMetAP2. Each 50 μL assay solution, contained 50 mM Hepes.Na⁺ (pH 7.5), 100 mM NaCl, 10-100 nM purified hMetAP2 enzyme, and varying amounts of Met-AMC (in 3% DMSO aqueous solution) or Met-pNA. Assays were initiated with the addition of substrate and the initial rates were corrected for the background rate determined in the absence of hMetAP2.

Coupled Spectrophotometric Assays of hMetAP2:

The methionine aminopeptidase activity of hMetAP2 can also be measured spectrophotometrically by monitoring the free L-amino acid formation. The release of N-terminal methionine from a tripeptide (Met-Ala-Ser, Sigma) or a tetrapeptide (Met-Gly-Met-Met, Sigma) substrate was assayed using the L-amino acid oxidase (AAO)/horse radish peroxidase (HRP) couple (eq. 1-3a,b). The formation of hydrogen peroxide (H₂O₂) was continuously monitored at 450 nm (absorbance increase of o-Dianisidine (Sigma) upon oxidation, Δε=15,300 M⁻¹ cm⁻¹)² and 30° C. in a 96- or 384-well plate reader by a method adapted from Tsunasawa, S. et al.(1997) (eq. 3a). Alternatively, formation of H₂O₂ was followed by monitoring the fluorescence emission increase at 587 nm (Δε=54,000 M⁻¹ cm⁻¹, λₑₓ=563 nm, slit width for both excitation and emission was 1.25 mm) and 30° C. using Amplex Red (Molecular Probes, Inc) (Zhou, M. et al. (1997) Anal. Biochem. 253, 162) (eq. 3b). In a total volume of 50 μL, a typical assay contained 50 mM HepesNa⁺, pH 7.5, 100 mM NaCl, 10 μM CoCl₂, 1 mM o-Dianisidine or 50 μM Amplex Red, 0.5 units of HRP (Sigma), 0.035 unit of AAO (Sigma), 1 nM hMetAP2, and varying amounts of peptide substrates. Assays were initiated by the addition of hMetAP2 enzyme, and the rates were corrected for the background rate determined in the absence of hMetAP2. 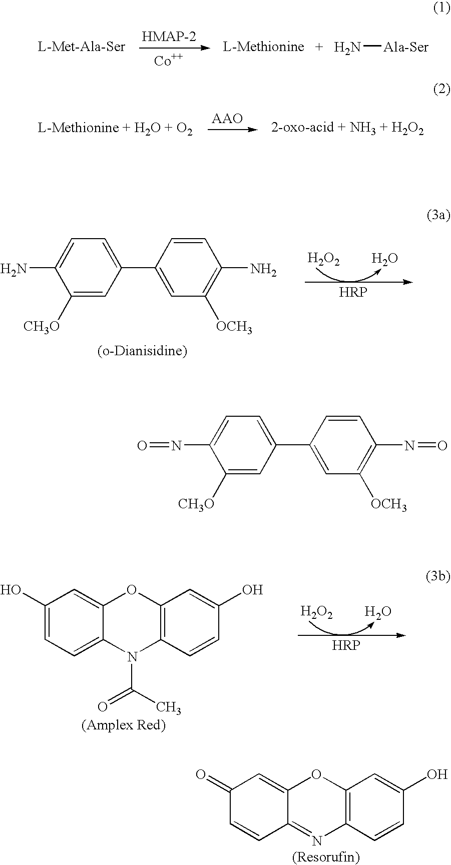
Kinetic Data Analysis:

Data were fitted to the appropriate rate equations using Grafit computer software. Initial velocity data conforming to Michaelis-Menton kinetics were fitted to eq. 4. Inhibition patterns conforming to apparent competitive and non-competitive inhibition were fitted to eq. 5 and eq. 6, respectively.
v=VA/(Kₐ+A) (4)
v=VA/[Kₐ(1+I/Kᵢₛ)+A] (5)
v=VA/[Kₐ(1+I/Kᵢₛ)+A(1+I/Kᵢᵢ)] (6)
In eqs. 4-6, v is the initial velocity, V is the maximum velocity, Kₐ is the apparent Michaelis constant, I is the inhibitor concentration, and A is the concentration of variable substrates. The nomenclature used in the rate equations for inhibition constants is that of Cleland (1963), in which Kᵢₛ and Kᵢᵢ represent the apparent slope and intercept inhibition constants, respectively.
Cell Growth Inhibition Assays

The ability of MetAP2 inhibitors to inhibit cell growth was assessed by the standard XTT microtitre assay. XTT, a dye sensitive to the pH change of mitochondria in eukaryotic cells, is used to quantify the viability of cells in the presence of chemical compounds. Cells seeded at a given number undergo approximately two divisions on average in the 72 hours of incubation. In the absence of any compound, this population of cells is in exponential growth at the end of the incubation period; the mitochondrial activity of these cells is reflected in the spectrophotometric readout (A₄₅₀). Viability of a similar cell population in the presence of a given concentration of compound is assessed by comparing the A₄₅₀ reading from the test well with that of the control well. Flat-bottomed 96-well plates are seeded with appropriate numbers of cells (1-4×10³ cells/well in a volume of 200 ul) from trypsinized exponentially growing cultures. To “blank” wells is added growth medium only. Cells are incubated overnight to permit attachment. Next day, medium from wells that contain cells is replaced with 180 ul of fresh medium. Appropriate dilutions of test compounds are added to the wells, final DMSO concentration in all wells being 0.2%. Cells plus compound are incubated for an additional 72 hr at 37° C. under the normal growth conditions of the cell line used. Cells are then assayed for viability using standard XTT/PMS (prepared immediately before use: 8 mg XTT (Sigma X-4251) per plate is dissolved in 100 ul DMSO. 3.9 ml H₂O is added to dissolve XTT and 20 ul of PMS stock solution (30 mg/ml) is added from frozen aliquoted stock solution (10 mg of PMS (phenazine methosulfate, Sigma P-9625) in 3.3 ml PBS without cations. These stocks are frozen at −20° C. until use). 50 μl of XTT/PMS solution is added to each well and plates incubated for 90 minutes (time required may vary according to cell line, etc.) at 37° C. until A₄₅₀ is >1.0. Absorbance at 450 nM is determined using a 96-well UV plate reader. Percent viability of cells in each well is calculated from these data (having been corrected for background absorbance). IC₅₀ is that concentration of compound that reduces cell viability to 50% control (untreated) viability.

The compounds of this invention show MetAP2 inhibitor activity having IC₅₀ values in the range of 0.0001 to 100 uM. The full structure/activity relationship has not yet been established for the compounds of this invention. However, given the disclosure herein, one of ordinary skill in the art can utilize the present assays in order to determine which compounds of this invention are inhibitors of MetAP2 and which bind thereto with an IC₅₀ value in the range of 0.0001 to 100 uM.

All publications, including, but not limited to, patents and patent applications cited in this specification, are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, given the preceding description, utilize the present invention to its fullest extent. Therefore any examples are to be construed as merely illustrative and not a limitation on the scope of the present invention in any way. The embodiments of the invention in which an exclusive property or privilege is claimed are defined as follows.

1. A compound of Formula (I): 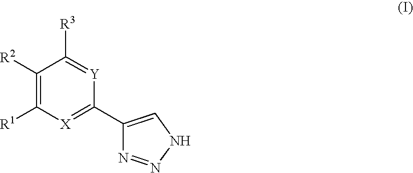
wherein:
X and Y are each independently selected from the group consisting of N, CH and CR, provided that X and Y are not both CR;
R is selected from the group consisting of: hydrogen, halogen, C₁-C₆ alkyl, C₁-C₆ haloalkyl, C₁-C₆ alkoxy, C₁-C₆ haloalkoxy, Ph-C₀-C₆ alkoxy, Het-C₀-C₆ alkoxy, hydroxyl, amino, (Ph-C₁-C₄ alkyl-)(C₁-C₄ alkyl)N—, (Ph-C₁-C₄ alkyl)HN—, (Het-C₁-C₄ alkyl-)(C₁-C₄ alkyl)N—, (Het-C₁-C₄ alkyl)HN—, (C₁-C₄ alkyl)(C₁-C₄ alkyl)N—, (C₁-C₄ alkyl)HN—, (C₁-C₄ alkyl)(C₁-C₄ alkyl)N—C₁-C₄ alkyl-, and (C₁-C₄ alkyl)HN—C₁-C₄ alkyl-;
R¹ is selected from the group consisting of: hydrogen, halogen, C₁-C₆ alkyl, C₁-C₆ haloalkyl, C₁-C₆ alkoxy, C₁-C₆ haloalkoxy, Ph-C₀-C₆ alkoxy, Het-C₀-C₆ alkoxy, (Ph-C₁-C₄ alkyl-)(C₁-C₄ alkyl)N—, (Ph-C₁-C₄ alkyl)HN—, (Het-C₁-C₄ alkyl-)(C₁-C₄ alkyl)N—, (Het-C₁-C₄ alkyl)HN—, (C₁-C₄ alkyl)(C₁-C₄ alkyl)N—, (C₁-C₄ alkyl)HN—, (C₁-C₄ alkyl)(C₁-C₄ alkyl)N—C₁-C₄ alkyl-, and (C₁-C₄ alkyl)HN—C₁-C₄ alkyl-;
R² and R³ are each independently selected from the group consisting of: hydrogen, halogen, C₁-C₆ alkyl, C₁-C₆ haloalkyl, C₁-C₆ alkoxy, C₁-C₆ haloalkoxy, hydroxyl, amino, (C₁-C₄ alkyl)HN— and (C₁-C₄ alkyl)(C₁-C₄ alkyl)N—;
wherein a (C₁-C₄ alkyl)N— moiety of any of the above (C₁-C₄ alkyl)(C₁-C₄ alkyl)N—, (C₁-C₄ alkyl)HN—, (C₁-C₄ alkyl)(C₁-C₄ alkyl)N—C₁-C₄ alkyl-, and (C₁-C₄ alkyl)HN—C₁-C₄ alkyl- is unsubstituted or substituted on carbon (e.g., substituted on (C₁-C₄ alkyl), not on N—) by a substituent selected from C₁-C₄ alkoxy, hydroxyl, hydroxy-C₁-C₄ alkyl-, C₁-C₄ alkylC(O)O—, C₁-C₄ alkoxyC(O)—, HOC(O)—C₁-C₄ alkoxy-, and (C₁-C₄ alkoxy)C(O)C₁-C₄ alkoxy-;
and wherein said Ph and Het are unsubstituted or substituted by one or more substituents independently selected from halogen, C₁-C₄ alkyl, C₁-C₄ alkoxy, C₁-C₄ haloalkyl, C₁-C₄ haloalkoxy, hydroxyl, hydroxy-C₁-C₄ alkyl-, C₁-C₄ alkylC(O)O—, C₁-C₄ alkoxyC(O)—, HOC(O)—C₁-C₄ alkoxy-, (C₁-C₄ alkoxy)C(O)C₁-C₄ alkoxy-, amino, (C₁-C₄ alkyl)HN—, (C₁-C₄ alkyl)(C₁-C₄ alkyl)N—, (C₁-C₄ alkyl)HN—C₁-C₄ alkyl-, and (C₁-C₄ alkyl)(C₁-C₄ alkyl)N—C₁-C₄ alkyl-;
or any two adjacent R, R¹, R² or R³ groups, taken together with the atoms to which they are attached, form a 6-membered carbocyclic aromatic ring, wherein said 6-membered carbocyclic aromatic ring is unsubstituted or substituted with one or more substituents independently selected from halogen, C₁-C₄ alkyl, C₁-C₄ alkoxy, C₁-C₄ haloalkyl and C₁-C₄ haloalkoxy;
provided that the compound of Formula (I) is not: 2-(1H-1,2,3-triazol-4-yl)-pyridine, 4-phenyl-1H-1,2,3-triazole, 4-(1H-1,2,3-triazol-4-yl)-phenol, 4-(4-fluorophenyl)-1H-1,2,3-triazole, 4-(3-bromophenyl)-1H-1,2,3-triazole, 4-(3-bromo-4-(trifluoromethoxy)phenyl)-1H-1,2,3-triazole, 4-(3-methylphenyl)-1H-1,2,3-triazole, 4-(4-methylphenyl)-1H-1,2,3-triazole, 4-(4-bromophenyl)-1H-1,2,3-triazole, 4-(3-chlorophenyl)-1H-1,2,3-triazole, 4-(4-chlorophenyl)-1H-1,2,3-triazole, 4-(4-ethylphenyl)-1H-1,2,3-triazole, 4-(3,5-dichlorophenyl)-1H-1,2,3-triazole, 4-[4-(t-butyl)phenyl]-1H-1,2,3-triazole, 4-[4-methoxyphenyl]-1H-1,2,3-triazole, 4-(2-napthyl)-1H-1,2,3-triazole, 4-(1H-1,2,3-triazol-4-yl)-aniline, 2-chloro-4-(1H-1,2,3,-triazol-4-yl)-aniline or N,N-dimethyl-4-(1H-1,2,3-triazol-4-yl)-benzylamine;
or a tautomer, pharmaceutically active salt or solvate thereof.

2. A pharmaceutical composition comprising a compound of Formula (I) according to claim 1 and a pharmaceutically acceptable carrier.

3. A process for making a compound of Formula (I) according to claim 1, said process comprising:
a) carbon homologation of an aldehyde to provide a compound having the formula: 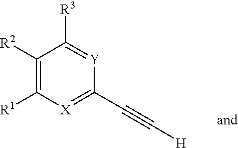
b) azide cycloaddition to the product of step a) to provide the compound of Formula (I), wherein X, Y, R¹, R² and R³ are defined as in claim 1.

4. A process for making a compounds of Formula (I) according to claim 1, said process comprising:
a) palladium mediated coupling of a bromide-containing compound having the formula: 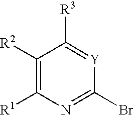
to provide a compound having the formula: 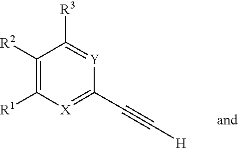
b) azide cycloaddition to the product of step a) to provide the compound of Formula (I), wherein Y, R¹, R² and R³ are defined as in claim 1.

5. (canceled)

6. A compound according to claim 1, selected from the group consisting of ethoxycarbonylmethyl-(3-[1H-1,2,3-triazol-4-yl]phenyl)-amine, (2-methoxy-benzyl)-[3-(1H-1,2,3-trizol-4-yl)phenyl]-amine, 5-hydroxymethyl-2-methyl-4-{[3-(1H-1,2,3-triazol-4-yl)-phenylamino]-methyl}-pyridin-3-ol, (4-acetoxy-3-methoxy-benzyl)-[3-(1H-1,2,3-triazol-4-yl)-phenyl]-amine, (2-carboxymethoxy-benzyl)-[3-(1H-1,2,3-triazol-4-yl)-phenyl]-amine, furan-2-ylmethyl-(4-bromo-3-[1H-1,2,3-triazol-4-yl]phenyl)amine, 2-(5-bromo-1H-1,2,3-triazol-4-yl)-pyridine, 3-(1H-1,2,3-triazol-4-yl)-isoquinoline, 4-(3,4-dibromophenyl)-1H-1,2,3-triazole, 4-(4-iodophenyl)-1H-1,2,3-triazole, 4-(3-phenoxy-phenyl)-1H-1,2,3-triazole, 4-(3-benzyloxy-phenyl)-1H-1,2,3-triazole, 4-(2-phenoxy-phenyl)-1H-1,2,3-triazole, 4-(2,4,5-tribromophenyl)-1H-1,2,3-triazole, 2-(1H-1,2,3-triazol-4-yl)-4-trifluoromethyl-pyridine, 4-ethyl-2-(1H-1,2,3-triazol-4-yl)-pyridine, 3-methyl-2-(1H-1,2,3-triazol-4-yl)-pyridine, 5-bromo-2-(1H-1,2,3-triazol-4-yl)-pyridine, 2-methoxy-6-(1H-1,2,3-triazol-4-yl)-pyridine, 2-(1H-1,2,3-triazol-4-yl)-pyrimidine, 4,5-dimethyl-2-(1H-1,2,3-triazol-4-yl)-pyridine, 2,4-dimethyl-6-(1H-1,2,3-triazol-4-yl)-pyridine, and 4-(2-hydroxyphenyl)-1H-1,2,3-triazole, or a tautomer, pharmaceutically active salt or solvate thereof.

7. A compound according to claim 1, selected from the group consisting of furan-2-ylmethyl-(4-bromo-3-[1H-1,2,3-triazol-4-yl]phenyl)amine, 3-(1H-1,2,3-triazol-4-yl)-isoquinoline, 4-(3,4-dibromophenyl)-1H-1,2,3-triazole, 4-(4-iodophenyl)-1H-1,2,3-triazole, 4-(3-phenoxy-phenyl)-1H-1,2,3-triazole, 4-(2-phenoxy-phenyl)-1H-1,2,3-triazole, 2-(1H-1,2,3-triazol-4-yl)-4-trifluoromethyl-pyridine, 4-ethyl-2-(1H-1,2,3-triazol-4-yl)-pyridine, 3-methyl-2-(1H-1,2,3-triazol-4-yl)-pyridine, 5-bromo-2-(1H-1,2,3-triazol-4-yl)-pyridine, 2-(1H-1,2,3-triazol-4-yl)-pyrimidine, and 2,4-dimethyl-6-(1H-1,2,3-triazol-4-yl)-pyridine, or a tautomer, pharmaceutically active salt or solvate thereof.

8. A method of inhibiting MetAP2 in mammals, comprising administering to a mammal in need of such inhibition, an effective amount of a compound of Formula (II): 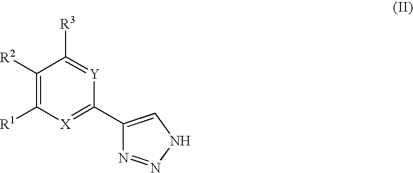
wherein:
X and Y are each independently selected from the group consisting of N, CH and CR, provided that X and Y are not both CR;
R is selected from the group consisting of: hydrogen, halogen, C₁-C₆ alkyl, C₁-C₆ haloalkyl, C₁-C₆ alkoxy, C₁-C₆ haloalkoxy, Ph-C₀-C₆ alkoxy, Het-C₀-C₆ alkoxy, hydroxyl, amino, (Ph-C₁-C₄ alkyl-)(C₁-C₄ alkyl)N—, (Ph-C₁-C₄ alkyl)HN—, (Het-C₁-C₄ alkyl-)(C₁-C₄ alkyl)N—, (Het-C₁-C₄ alkyl)HN—, (C₁-C₄ alkyl)(C₁-C₄ alkyl)N—, (C₁-C₄ alkyl)HN—, (C₁-C₄ alkyl)(C₁-C₄ alkyl)N—C₁-C₄ alkyl-, and (C₁-C₄ alkyl)HN—C₁-C₄ alkyl-;
R¹ is selected from the group consisting of: hydrogen, halogen, C₁-C₆ alkyl, C₁-C₆ haloalkyl, C₁-C₆ alkoxy, C₁-C₆ haloalkoxy, Ph-C₀-C₆ alkoxy, Het-C₀-C₆ alkoxy, (Ph-C₁-C₄ alkyl-)(C₁-C₄ alkyl)N—, (Ph-C₁-C₄ alkyl)HN—, (Het-C₁-C₄ alkyl-)(C₁-C₄ alkyl)N—, (Het-C₁-C₄ alkyl)HN—, (C₁-C₄ alkyl)(C₁-C₄ alkyl)N—, (C₁-C₄ alkyl)HN—, (C₁-C₄ alkyl)(C₁-C₄ alkyl)N—C₁-C₄ alkyl-, and (C₁-C₄ alkyl)HN—C₁-C₄ alkyl-;
R² and R³ are each independently selected from the group consisting of: hydrogen, halogen, C₁-C₆ alkyl, C₁-C₆ haloalkyl, C₁-C₆ alkoxy, C₁-C₆ haloalkoxy, hydroxyl, amino, (C₁-C₄ alkyl)HN— and (C₁-C₄ alkyl)(C₁-C₄ alkyl)N—;
wherein a (C₁-C₄ alkyl)N— moiety of any of the above (C₁-C₄ alkyl)(C₁-C₄ alkyl)N—, (C₁-C₄ alkyl)HN—, (C₁-C₄ alkyl)(C₁-C₄ alkyl)N—C₁-C₄ alkyl-, and (C₁-C₄ alkyl)HN—C₁-C₄ alkyl- is unsubstituted or substituted on carbon (e.g., substituted on (C₁-C₄ alkyl), not on N—) by a substituent selected from C₁-C₄ alkoxy, hydroxyl, hydroxy-C₁-C₄ alkyl-, C₁-C₄ alkylC(O)O—, C₁-C₄ alkoxyC(O)—, HOC(O)—C₁-C₄ alkoxy-, and (C₁-C₄ alkoxy)C(O)C₁-C₄ alkoxy-;
and wherein said Ph and Het are unsubstituted or substituted by one or more substituents independently selected from halogen, C₁-C₄ alkyl, C₁-C₄ alkoxy, C₁-C₄ haloalkyl, C₁-C₄ haloalkoxy, hydroxyl, hydroxy-C₁-C₄ alkyl-, C₁-C₄ alkylC(O)O—, C₁-C₄ alkoxyC(O)—, HOC(O)—C₁-C₄ alkoxy-, (C₁-C₄ alkoxy)C(O)C₁-C₄ alkoxy-, amino, (C₁-C₄ alkyl)HN—, (C₁-C₄ alkyl)(C₁-C₄ alkyl)N—, (C₁-C₄ alkyl)HN—C₁C₄ alkyl-, and (C₁-C₄ alkyl)(C₁-C₄ alkyl)N—C₁-C₄ alkyl-;
or any two adjacent R, R¹, R² or R³ groups, taken together with the atoms to which they are attached, form a 6-membered carbocyclic aromatic ring, wherein said 6-membered carbocyclic aromatic ring is unsubstituted or substituted with one or more substituents independently selected from halogen, C₁-C₄ alkyl, C₁-C₄ alkoxy, C₁-C₄ haloalkyl and C₁-C₄ haloalkoxy;
or a tautomer, pharmaceutically active salt or solvate thereof.

9. The method according to claim 8, wherein the compound of Formula (II) is selected from ethoxycarbonylmethyl-(3-[1H-1,2,3-triazol-4-yl]phenyl)-amine, (2-methoxy-benzyl)-[3-(1H-1,2,3-trizol-4-yl)phenyl]-amine, 5-hydroxymethyl-2-methyl-4-{[3-(1H-1,2,3-triazol-4-yl)-phenylamino]-methyl}-pyridin-3-ol, (4-acetoxy-3-methoxy-benzyl)-[3-(1H-1,2,3-triazol-4-yl)-phenyl]-amine, (2-carboxymethoxy-benzyl)-[3-(1H-1,2,3-triazol-4-yl)-phenyl]-amine, furan-2-ylmethyl-(4-bromo-3-[1H-1,2,3-triazol-4-yl]phenyl)amine, 2-(5-bromo-1H-1,2,3-triazol-4-yl)-pyridine, 3-(1H-1,2,3-triazol-4-yl)-isoquinoline, 4-dibromophenyl)-1H-1,2,3-triazole, 4-(3-bromophenyl)-1H-1,2,3-triazole, 4-(4-iodophenyl)-1H-1,2,3-triazole, 4-(3-phenoxy-phenyl)-1H-1,2,3-triazole, 4-(3-benzyloxy-phenyl)-1H-1,2,3-triazole, 4-(2-phenoxy-phenyl)-1H-1,2,3-triazole, 4-(2,4,5-tribromophenyl)-1H-1,2,3-triazole, 2-(1H-1,2,3-triazol-4-yl)-4-trifluoromethyl-pyridine, 4-ethyl-2-(1H-1,2,3-triazol-4yl)-pyridine, 3-methyl-2-(1H-1,2,3-triazol-4-yl)-pyridine, 5-bromo-2-(1H-1,2,3-triazol-4-yl)-pyridine, 2-methoxy-6-(1H-1,2,3-triazol-4-yl)-pyridine, 2-(1H-1,2,3-triazol-4-yl)-pyrimidine, 4,5-dimethyl-2-(1H-1,2,3-triazol-4-yl)-pyridine, 2,4-dimethyl-6-(1H-1,2,3-triazol-4-yl)-pyridine, 4-(2-hydroxyphenyl)-1H-1,2,3-triazole, 2-(1H-1,2,3-triazol-4-yl)-pyridine, 4-phenyl-1H-1,2,3-triazole, 4-(1H-1,2,3-triazol-4-yl)-phenol, 4-(4-fluorophenyl)-1H-1,2,3-triazole, 4-(3-bromophenyl)-1H-1,2,3-triazole, 4-(3-bromo-4-(trifluoromethoxy)phenyl)-1H-1,2,3-triazole, 4-(3-methylphenyl)-1H-1,2,3-triazole, 4-(4-methylphenyl)-1H-1,2,3-triazole, 4-(4-bromophenyl)-1H-1,2,3-triazole, 4-(3-chlorophenyl)-1H-1,2,3-triazole, 4-(4-chlorophenyl)-1H-1,2,3-triazole, 4-(4-ethylphenyl)-1H-1,2,3-triazole, 4-(3,5-dichlorophenyl)-1H-1,2,3-triazole, 4-[4-(t-butyl)phenyl]-1H-1,2,3-triazole, 4-[4-methoxyphenyl]-1H-1,2,3-triazole, 4-(2-napthyl)-1H-1,2,3-triazole, 4-(1H-1,2,3-triazol-4-yl)-aniline, 2-chloro-4-(1H-1,2,3-triazol-4-yl)-aniline and N,N-dimethyl-4-(1H-1,2,3-triazol-4-yl)-benzylamine, or a tautomer, pharmaceutically acceptable salt or solvate thereof.

10. A method for treating a disease mediated by MetAP2 in mammals, comprising administering to a mammal in need of such treatment, an effective amount of a compound of Formula (II): 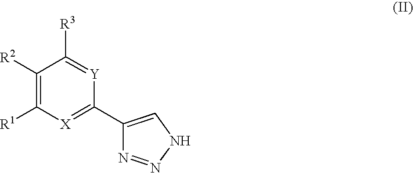
wherein:
X and Y are each independently selected from the group consisting of N, CH and CR, provided that X and Y are not both CR;
R is selected from the group consisting of: hydrogen, halogen, C₁-C₆ alkyl, C₁-C₆ haloalkyl, C₁-C₆ alkoxy, C₁-C₆ haloalkoxy, Ph-C₀-C₆ alkoxy, Het-C₀-C₆ alkoxy, hydroxyl, amino, (Ph-C₁-C₄ alkyl-)(C₁-C₄ alkyl)N—, (Ph-C₁-C₄ alkyl)HN—, (Het-C₁-C₄ alkyl-)(C₁-C₄ alkyl)N—, (Het-C₁-C₄ alkyl)HN—, (C₁-C₄ alkyl)(C₁-C₄ alkyl)N—, (C₁-C₄ alkyl)HN—, (C₁-C₄ alkyl)(C₁-C₄ alkyl)N—C₁-C₄ alkyl-, and (C₁-C₄ alkyl)HN—C₁-C₄ alkyl-;
R¹ is selected from the group consisting of: hydrogen, halogen, C₁-C₆ alkyl, C₁-C₆ haloalkyl, C₁-C₆ alkoxy, C₁-C₆ haloalkoxy, Ph-C₀-C₆ alkoxy, Het-C₀-C₆ alkoxy, (Ph-C₁-C₄ alkyl-)(C₁-C₄ alkyl)N—, (Ph-C₁-C₄ alkyl)HN—, (Het-C₁-C₄ alkyl-)(C₁-C₄ alkyl)N—, (Het-C₁-C₄ alkyl)HN—, (C₁-C₄ alkyl)(C₁-C₄ alkyl)N—, (C₁-C₄ alkyl)HN—, (C₁-C₄ alkyl)(C₁-C₄ alkyl)N—C₁-C₄ alkyl-, and (C₁-C₄ alkyl)HN—C₁-C₄ alkyl-;
R² and R³ are each independently selected from the group consisting of: hydrogen, halogen, C₁-C₆ alkyl, C₁-C₆ haloalkyl, C₁-C₆ alkoxy, C₁-C₆ haloalkoxy, hydroxyl, amnino, (C₁-C₄ alkyl)HN— and (C₁-C₄ alkyl)(C₁-C₄ alkyl)N—;
wherein a (C₁-C₄ alkyl)N— moiety of any of the above (C₁-C₄ alkyl)(C₁-C₄ alkyl)N—, (C₁-C₄ alkyl)HN—, (C₁-C₄ alkyl)(C₁-C₄ alkyl)N—C₁-C₄ alkyl-, and (C₁-C₄ alkyl)HN—C₁-C₄ alkyl- is unsubstituted or substituted on carbon (e.g., substituted on (C₁-C₄ alkyl), not on N—) by a substituent selected from C₁-C₄ alkoxy, hydroxyl, hydroxy-C₁-C₄ alkyl-, C₁-C₄ alkylC(O)O—, C₁-C₄ alkoxyC(O)—, HOC(O)—C₁-C₄ alkoxy-, and (C₁-C₄ alkoxy)C(O)C₁-C₄ alkoxy-;
and wherein said Ph and Het are unsubstituted or substituted by one or more substituents independently selected from halogen, C₁-C₄ alkyl, C₁-C₄ alkoxy, C₁-C₄ haloalkyl, C₁-C₄ haloalkoxy, hydroxyl, hydroxy-C₁-C₄ alkyl-, C₁-C₄ alkylC(O)O—, C₁-C₄ alkoxyC(O)—, HOC(O)—C₁-C₄ alkoxy-, (C₁-C₄ alkoxy)C(O)C₁-C₄ alkoxy-, amino, (C₁-C₄ alkyl)HN—, (C₁-C₄ alkyl)(C₁-C₄ alkyl)N—, (C₁-C₄ alkyl)HN—C₁-C₄ alkyl-, and (C₁-C₄ alkyl)(C₁-C₄ alkyl)N—C₁-C₄ alkyl-;
or any two adjacent R, R¹, R² or R³ groups, taken together with the atoms to which they are attached, form a 6-membered carbocyclic aromatic ring, wherein said 6-membered carbocyclic aromatic ring is unsubstituted or substituted with one or more substituents independently selected from halogen, C₁-C₄ alkyl, C₁-C₄ alkoxy, C₁-C₄ haloalkyl and C₁-C₄ haloalkoxy;
or a tautomer, pharmaceutically active salt or solvate thereof.

11. The method according to claim 10, wherein the compound of Formula (II) is selected from ethoxycarbonylmethyl-(3-[1H-1,2,3-triazol-4-yl]phenyl)-amine, (2-methoxy-benzyl)-[3-(1H-1,2,3-trizol-4-yl)phenyl]-amine, 5-hydroxymethyl-2-methyl-4-{[3-(1H-1,2,3-triazol-4-yl)-phenylamino]-methyl}-pyridin-3-ol, (4-acetoxy-3-methoxy-benzyl)-[3-(1H-1,2,3-triazol-4-yl)-phenyl]-amine, (2-carboxymethoxy-benzyl)-[3-(1H-1,2,3-triazol-4-yl)-phenyl]-amine, furan-2-ylmethyl-(4-bromo-3-[1H-1,2,3-triazol-4-yl]phenyl)amine, 2-(5-bromo-1H-1,2,3-triazol-4-yl)-pyridine, 3-(1H-1,2,3-triazol-4-yl)-isoquinoline, 4-(3,4-dibromophenyl)-1H-1,2,3-triazole, 4-(3-bromophenyl)-1H-1,2,3-triazole, 4-(4-iodophenyl)-1H-1,2,3-triazole, 4-(3-phenoxy-phenyl)-1H-1,2,3-triazole, 4-(3-benzyloxy-phenyl)-1H-1,2,3-triazole, 4-(2-phenoxy-phenyl)-1H-1,2,3-triazole, 4-(2,4,5-tribromophenyl)-1H-1,2,3-triazole, 2-(1H-1,2,3-triazol-4-yl)-4-trifluoromethyl-pyridine, 4-ethyl-2-(1H-1,2,3-triazol-4-yl)-pyridine, 3-methyl-2-(1H-1,2,3-triazol-4-yl)-pyridine, 5-bromo-2-(1H-1,2,3-triazol-4-yl)-pyridine, 2-methoxy-6-(1H-1,2,3-triazol-4-yl)-pyridine, 2-(1H-1,2,3-triazol-4-yl)-pyrimidine, 4,5-dimethyl-2-(1H-1,2,3-triazol-4-yl)-pyridine, 2,4-dimethyl-6-(1H-1,2,3-triazol-4-yl)-pyridine, 4-(2-hydroxyphenyl)-1H-1,2,3-triazole, 2-(1H-1,2,3-triazol-4-yl)-pyridine, 4-phenyl-1H-1,2,3-triazole, 4-(1H-1,2,3-triazol-4-yl)-phenol, 4-(4-fluorophenyl)-1H-1,2,3-triazole, 4-(3-bromophenyl)-1H-1,2,3-triazole, 4-(3-bromo-4-(trifluoromethoxy)phenyl)-1H-1,2,3-triazole, 4-(3-methylphenyl)-1H-1,2,3-triazole, 4-(4-methylphenyl)-1H-1,2,3-triazole, 4-(4-bromophenyl)-1H-1,2,3-triazole, 4-(3-chlorophenyl)-1H-1,2,3-triazole, 4-(4-chlorophenyl)-1H-1,2,3-triazole, 4-(4-ethylphenyl)-1H-1,2,3-triazole, 4-(3,5-dichlorophenyl)-1H-1,2,3-triazole, 4-[4-(t-butyl)phenyl]-1H-1,2,3-triazole, 4-[4-methoxyphenyl]-1H-1,2,3-triazole, 4-(2-napthyl)-1H-1,2,3-triazole, 4-(1H-1,2,3-triazol-4-yl)-aniline, 2-chloro-4-(1H-1,2,3-triazol-4-yl)-aniline and N,N-dimethyl-4-(1H-1,2,3-triazol-4-yl)-benzylamine, or a tautomer, pharmaceutically acceptable salt or solvate thereof.

12. The compound according to claim 1, wherein
when both X and Y are CH: R¹ is Ph-C₀-C₆ alkoxy, (Het-C₁-C₄ alkyl)HN—, or (Ph-C₁-C₄ alkyl)HN—, wherein Ph is unsubstituted or substituted by C₁-C₄ alkoxy and R² and R³ are hydrogen; or R¹ and R² are each independently selected from halogen or C₁-C₆ alkyl and R³is hydrogen, wherein when R¹ and R² are both halogen or are both C₁-C₆ alkyl, each of said halogen or C₁-C₆ alkyl is the same or different; or
when Y is CR and X is CH: R is Ph-C₀-C₆ alkoxy and R¹, R² and R³ are hydrogen; or R is halogen and R¹ is selected from hydrogen, halogen or (Het-C₁-C₄ alkyl)HN—, R² is selected from hydrogen or halogen and R³ is hydrogen, wherein R¹ and R² are not both hydrogen; or
when Y is N and X is CH: R¹ is C₁-C₆ alkyl or C₁-C₆ haloalkyl and R² and R³ are hydrogen; or R² is halogen or C₁-C₆ alkyl and R¹ and R³ are hydrogen; or R¹ and R³ are each C₁-C₆ alkyl and R² is hydrogen; or R¹ and R² taken together with the atoms to which they are attached form a 6-membered aromatic ring and R³ is hydrogen; or
when Y is N and X is CR: R is C₁-C₆ alkyl, R¹, R² and R³ are hydrogen; or
when Y is N and X is N: R¹, R² and R³ are hydrogen;
or a tautomer, pharmaceutically acceptable salt or solvate thereof.

13. The method according to claim 8, comprising administering a compound of Formula (II) wherein:
when both X and Y are CH: R¹ is Ph-C₀-C₆ alkoxy, (Het-C₁-C₄ alkyl)HN—, or (Ph-C₁-C₄ alkyl)HN—, wherein Ph is unsubstituted or substituted by C₁-C₄ alkoxy and R² and R³ are hydrogen; or R¹ is halogen or C₁-C₆ alkyl and R² and R³ are hydrogen; or R² is halogen or C₁-C₆ alkyl and R¹ and R³ are hydrogen; or R¹ and R² are each independently selected from halogen or C₁-C₆ alkyl and R³ is hydrogen, wherein when R¹ and R² are both halogen or are both C₁-C₆ alkyl, each of said halogen or C₁-C₆ alkyl is the same or different; or
when Y is CR and X is CH: R is Ph-C₀-C₆ alkoxy and R¹, R² and R³ are hydrogen; or R is halogen and R¹ is selected from hydrogen, halogen or (Het-C₁-C₄ alkyl)HN—, R² is selected from hydrogen or halogen and R³is hydrogen, wherein R¹ and R² are not both hydrogen; or
when Y is N and X is CH: R¹ is C₁-C₆ alkyl or Cl-C₆ haloalkyl and R² and R³ are hydrogen; or R² is halogen or C₁-C₆ alkyl and R¹ and R³ are hydrogen; or R¹ and R³ are each C₁-C₆ alkyl and R² is hydrogen; or R¹ and R² taken together with the atoms to which they are attached form a 6-membered aromatic ring and R³ is hydrogen; or
when Y is N and X is CR: R is C₁-C₆ alkyl, R¹, R² and R³ are hydrogen; or
when Y is N and X is N: R¹, R² and R³ are hydrogen;
or a tautomer, pharmaceutically acceptable salt or solvate thereof.

14. The method according to claim 10, comprising administering a compound of Formula (II) wherein
when both X and Y are CH: R¹ is Ph-C₀-C₆ alkoxy, (Het-C₁-C₄ alkyl)HN—, or (Ph-C₁-C₄ alkyl)HN—, wherein Ph is unsubstituted or substituted by C₁-C₄ alkoxy and R² and R³ are hydrogen; or R¹ is halogen or C₁-C₆ alkyl and R² and R³ are hydrogen; or R² is halogen or C₁-C₆ alkyl and R¹ and R³ are hydrogen; or R¹ and R² are each independently selected from halogen or C₁-C₆ alkyl and R³ is hydrogen, wherein when R¹ and R² are both halogen or are both C₁-C₆ alkyl, each of said halogen or C₁-C₆ alkyl are the same or different; or
when Y is CR and X is CH: R¹ is Ph-C₀-C₆ alkoxy and R¹, R² and R³ are hydrogen; or R is halogen and R¹ is selected from hydrogen, halogen or (Het-C₁-C₄ alkyl)HN—, R² is selected from hydrogen or halogen and R³ is hydrogen, wherein R¹ and R² are not both hydrogen; or
when Y is N and X is CH: R¹ is C₁-C₆ alkyl or C₁-C₆ haloalkyl and R² and R³ are hydrogen; or R² is halogen or C₁-C₆ alkyl and R¹ and R³ are hydrogen; or R¹ and R³ are each C₁-C₆ alkyl and R² is hydrogen; or R¹ and R² taken together with the atoms to which they are attached form a 6-membered aromatic ring and R³ is hydrogen; or
when Y is N and X is CR: R is C₁-C₆ alkyl, R¹, R² and R³ are hydrogen; or
when Y is N and X is N: R¹, R² and R³ are hydrogen;
or a tautomer, pharmaceutically acceptable salt or solvate thereof.
